SEQUENCE LISTING (16 sequences)

SEQ ID NO: 1 - protein, 139 aa, sesame
MAEHYGQQQQTRAPHLQLQPRAQRVVKAATAVTAGGSLLVLSGLTLAGTVIALTIATPLLVIFSPVLVPAVITIFLLGAGFLASGGFGVAALSVLSWIYRYLTGKHPPGADQLESAKTKLASKAREMKDRAEQFSQQPV

SEQ ID NO: 2 - protein, 245 aa, sesame
MATHVLAAAAERNAALAPDAPLAPVTMERPVRTDLETSIPKPYMARGLVAPDMDHPNGTPGHVHDNLSVLQQHCAFFDQDDNGIIYPWETYSGLRQIGFNVIASLIMAIVINVALSYPTLPGWIPSPFFPIYLYNIHKAKHGSDSGTYDTEGRYLPMNFENLFSKHARTMPDRLTLGELWSMTEANREAFDIFGWIASKMEWTLLYILARDQDGFLSKEAIRRCYDGSLFEYCAKMQRGAEDKMK

SEQ ID NO: 3 - protein, 58 aa, Artificial Sequence
  ZHer2 peptide
VDNKFNKEMRNAYWEIALLPNLNNQQKRAFIRSLYDDPSQSANLLAEAKKLNDAQAPK

SEQ ID NO: 4 - protein, 8 aa, human
CRGDMFGC

SEQ ID NO: 5 - protein, 40 aa, HIV-1
RIRLTGLNSGLGMLGISYGRGISYGRKKRRQRRRPPQTGG

SEQ ID NO: 6 - DNA, 114 bp, Artificial Sequence
  a linker gene
gccatggagatatcggaattaattcggatccgcctgaccggtctgaactcaggcctgaacgatattttcgaagctcagaaaatcgaatggcacgaggacactggtggctcgagc

SEQ ID NO: 7 - protein, 38 aa, Artificial Sequence
  a linker
AMEISELIRIRLTGLNSGLNDIFEAQKIEWHEDTGGSS

SEQ ID NO: 8 - DNA, 417 bp, sesame
atggctgagcattatggtcaacaacagcagaccagggcgcctcacctgcagctgcagccgcgcgcccagcgggtagtgaaggcggccaccgccgtgacagccggcggctcgcttctcgtcctctctggcctcactttagccggaactgttattgcgctcaccatcgccactccgctgcttgtgatctttagccccgttctggtgccggcggtcataaccattttcttgctgggtgcgggttttctggcatccggaggcttcggcgtggcggcgctgagtgtgctgtcgtggatttacagatatctgacagggaaacacccgccgggggcggatcagctggaatcggcaaagacgaagctggcgagcaaggcgcgagagatgaaggatagggcagagcagttctcgcagcagcctgtt

SEQ ID NO: 9 - DNA, 375 bp, Artificial Sequence
  ZHer2 gene
gttgataacaaattcaacaaagaaatgcgcaacgcgtactgggaaatcgcactgctgccgaacctgaacaaccagcagaaacgcgccttcatccgcagcctgtatgatgatccgagccagagcgcgaacctgctggcagaagcgaaaaaactgaatgatgcgcaggcgccgaaaggaaggatttcagaattcggatccgttgataacaaattcaacaaagaaatgcgcaacgcgtactgggaaatcgcactgctgccgaacctgaacaaccagcagaaacgcgccttcatccgcagcctgtatgatgatccgagccagagcgcgaacctgctggcagaagcgaaaaaactgaatgatgcgcaggcgccgaaaaag

SEQ ID NO: 10 - DNA, 24 bp, Artificial Sequence
  a linker gene
tcgatatcggaattaattcggatc

SEQ ID NO: 11 - protein, 8 aa, Artificial Sequence
  a linker
SISELIRI

SEQ ID NO: 12 - DNA, 735 bp, sesame
atggcaactcatgttttggctgctgcggcggagagaaatgctgcgttggcgccggacgccccgcttgctccggtgactatggagcgcccagtgcgcactgacttggagacttcgatcccgaagccctatatggcaagaggattggttgcacctgatatggatcaccccaacggaacaccaggccatgtgcatgataatttgagtgtgctgcaacagcattgtgctttctttgatcaggatgataacggaatcatctatccatgggagacttactctggacttcgccagattggtttcaatgtgatagcttcccttataatggctatcgtcattaatgtggcgctgagttatcctactctcccgggttggattccttctccttttttccccatatatttgtacaacatacacaaggccaaacatggaagcgactccggaacctatgatactgaaggaaggtacctacctatgaattttgagaacctgttcagcaagcatgcccggacaatgcccgataggctcactctaggggagctatggagcatgactgaagctaacagagaagcatttgacattttcggctggatcgcaagcaaaatggagtggactctcctctacattcttgcaagagaccaggacggtttcctgtcgaaagaagccatcaggcggtgttacgatggcagtttgttcgagtactgtgcaaagatgcaaaggggagccgaggacaagatgaaa

SEQ ID NO: 13 - DNA, 25 bp, Artificial Sequence
  a linker gene
gccatggagatatcggaattaattc

SEQ ID NO: 14 - protein, 8 aa, Artificial Sequence
  a linker
AMEISELI

SEQ ID NO: 15 - DNA, 24 bp, human
tgccgcggcgatatgtttggctgc

SEQ ID NO: 16 - DNA, 117 bp, HIV-1
cggatccgcctgaccggtctgaactcaggcctcggcatgctgggcattagctatggccgcggcattagctatggccgcaaaaaacgtcgtcagcgtcgtcgtccgccgcagaccggc

(19) United States
(12) Patent Application Publication
(10) Pub. No.: US 2011/0123448 A1
(43) Pub. Date: May 26, 2011

(54) OIL BODY CARRIERS, USES IN TARGET THERAPY AND/OR DETECTION OF THE SAME, AND FUSION PROTEINS COMPRISED THEREIN

(75) Inventors: Chung-Jen CHIANG, Taichung (TW); Yun-Peng CHAO, Taichung (TW); Tze-Cheng TZEN, Taichung (TW); Hesin-Fu WANG, Taichung (TW); Chih-Jung CHEN, Taichung (TW)

(73) Assignee: CHINA MEDICAL UNIVERSITY, TAICHUNG (TW)

(21) Appl. No.: 12/841,500

(22) Filed: Jul. 22, 2010

(30) Foreign Application Priority Data
Nov. 25, 2009 (TW) 098140119

(51) Int. Cl. A61K 9/14; C07K 14/00; C07K 16/00; C07K 14/415; A61K 31/01; A61K 31/12; A61K 31/437; A61K 31/56; A61K 47/44; A61P 35/00; A61K 49/00

(52) U.S. Cl. 424/9.1; 530/402; 530/387.3; 530/377; 530/378; 424/489; 514/762; 514/679; 514/283; 514/169; 514/281; 514/773; 977/773

(57) ABSTRACT

An oil body carrier, its uses in target therapy and/or detection, and a fusion protein comprised therein are provided. The oil body carrier comprises:

a) the fusion protein, comprising an oil body protein and a ligand peptide, an antibody peptide, a cell penetrating peptide, or combinations thereof, and

b) a lipid,

wherein the weight/volume (μg/μl) ratio of the fusion protein and the lipid is at least about 1/25, and the average particle size of the oil body carrier is about 10 nm to about 2,000 nm.

This application claims priority to Taiwan Patent Application No. 098140119 filed on Nov. 25, 2009.

### CROSS-REFERENCES TO RELATED APPLICATIONS

Not applicable.

### BACKGROUND OF THE INVENTION

1. Field of the Invention

The present invention relates to an oil body carrier, its uses, and a fusion protein comprised therein, particularly to uses of the oil body carrier in target therapy and/or detection.

2. Descriptions of the Related Art

Cancers, also known as malignant tumors, are cells that divide abnormally and prolifically throughout the body, resulting in the abnormality of physiological functions. Cancer is one of the major causes of human death because it cannot currently be cured.

Traditionally, a major method for treating cancer is to remove the malignant tumors from the body by surgery, or killing cancer cells with chemical drugs or radioactive rays. Unfortunately, surgery is often dangerous and difficult to perform, with the possibility that some cancerous cells cannot be completely removed. As a result, chemical drugs and radioactive rays may be used; however, this form of treatment has no specificity, and thus, during the treatment, other normal cells necessary for maintaining general physiological functions are usually killed, leading to many side effects, such as the decline of immunological functions (e.g., the decrease in the number of white blood cells), nausea, vomit, hair-loss, degradation of the absorption function of the intestine and stomach, anemia, etc.

It has been discovered that some special molecular biomarkers usually present on the surface of cancer cells, and based on this feature, a “target therapy” method has been developed for treating cancer over recent years. The principle of the target therapy method is to design a drug that can recognize the biomarkers specifically to block the activation of cancer cells or inhibit the growth of cancer cells efficiently for treatment. Because drugs designed for target therapy hit target cancer cells directly and precisely, the influence of the drugs on normal cells in the body is relatively lessened. Hence, compared with traditional methods, target therapy has advantages, such as low toxicity, low side effects, high efficiency, easy application, etc., and thus has already drawn much attention.

Many kinds of target therapy modes have been reported, one of which is using a pharmaceutical molecule that has a treating activity covered, linked, or embedded by a carrier, and applying the different kinds of mechanisms to “deliver” the carrier and the pharmaceutical molecule to target cancer cells specifically. This mode is generally called the “drug delivery system,” which can be seen in Morgillo et al., Resistance to epidermal growth factor receptor-targeted therapy. Drug Resist Updat. 2005, 8:298-310, which is entirely incorporated hereinto by reference. Generally, this mode is constructed by combining a carrier and a substance (which is usually called a “ligand”) that can specifically recognize biomarkers on the surface of cancer cells to give the carrier a targeting function. The substance can be proteins (e.g., antibodies), steroids, saccharides, compounds, etc.

Carriers currently known include high molecular polymer granules, micells, liposomes, virulent carriers, etc. (which can be seen in Kawano et al., Enhanced antitumor effect of camptothecin loaded in long-circulating polymeric micelles, J. Control. Release. 2006, 112: 329-332; Koshkina et al., Distribution of camptothecin after delivery as a liposome aerosol or following intramuscular injection in mice. Cancer Chemother Pharmacol. 1999, 44(3): 187-192; and Yang et al., Body distribution in mice of intravenous injected camptothecin solid lipid nanoparticles and targeting effect on brain, J. Control. Release. 1999, 59: 299-307, which are entirely incorporated hereinto by reference). However, these carriers have different disadvantages; for instance, some of the carriers have large particle sizes and are hard to be absorbed by the human body; some are toxic; and some are unstable in the blood or body fluid, and thus the application of these carriers is limited in practice.

In addition, when the above carriers and a substance that can recognize cancer cells are combined, complicated and time-consuming chemical synthesis procedures are usually required, which not only increases the manufacturing cost, but also causes environmental pollution. Therefore, in the field of the drug delivery system, there is still room for improvement, and a carrier that is small in particle size, nontoxic, stable, and/or easy to combine with a substance that can recognize cancer cells is still required.

The present invention meets the above requirements. By using techniques in molecular biology, the present invention provides an oil body carrier, and the preparation process thereof is simple. Pollution to the environment caused thereby is low or can even be negligible. Through relative in vivo and in vitro experiments, it was found that the oil body carrier of the present invention can be efficiently used in target therapy or detection, and has superior bio-compatibility.

### SUMMARY OF THE INVENTION

The primary objective of this invention is to provide a fusion protein, comprising an oil body protein and a ligand peptide, an antibody peptide, a cell penetrating peptide, or combinations thereof.

Another objective of this invention is to provide an oil body carrier, comprising the aforesaid fusion protein and a lipid, wherein the weight/volume (μg/μl) ratio of the fusion protein and the lipid is at least about 1/25, and the average particle size of the oil body carrier is about 10 nm to about 2,000 nm.

Yet a further objective of this invention is to provide a composition for target therapy and/or detection, comprising the above oil body carrier and a medicament, a signal molecule, or a combination thereof.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a drawing showing the structure of an oil body of a plant seed;

FIG. 2 illustrates a flowchart of the preparation of the oil body carrier of the present invention;

FIG. 3 presents electrophoresis pictures of the fusion proteins of the present invention;

FIG. 4A presents optical microscope images of the oil body carrier of the present invention comprising oleosin-Z_{Her2} peptide fusion protein;

FIG. 4B presents optical microscope images of the oil body carrier of the present invention comprising caleosin-Z_{Her2} peptide fusion protein;

FIGS. 5A and 5B present fluorescence microscope images of the oil body carriers of the present invention comprising oleosin-Z_{Her2} peptide fusion protein and caleosin-Z_{Her2} peptide fusion protein, respectively;

FIG. 6 presents curve diagrams illustrating the turbidity of the oil body carriers of the present invention;

FIG. 7 presents diagrams showing the distribution of the particle size of the oil body carriers of the present invention;

FIG. 8 presents curve diagrams illustrating the ratio and the embedded rate of fusion protein and lipid in the oil body carriers of the present invention;

FIGS. 9 and 10 present fluorescence microscope images of tumor cells comprising the oil body carriers of the present invention;

FIG. 11 presents fluorescence microscope images of MCF7/HER18 cells comprising different MOI values of the oil body carrier containing oleosin-Z_{Her2} peptide fusion protein;

FIG. 12 presents fluorescence microscope images of SKOV3 cells comprising different MOI values of the oil body carrier containing oleosin-Z_{Her2} peptide fusion protein;

FIG. 13 presents fluorescence microscope images of MCF7/HER18 cells comprising different MOI values of the oil body carrier containing caleosin-Z_{Her2} peptide fusion protein;

FIG. 14 presents fluorescence microscope images of SKOV3 cells comprising different MOI values of the oil body carrier containing caleosin-Z_{Her2} peptide fusion protein;

FIG. 15 presents fluorescence microscope images of MCF7 cells comprising different MOI values of the oil body carrier containing oleosin-TR peptide fusion protein;

FIG. 16 presents fluorescence microscope images of SKOV3 cells comprising different MOI values of the oil body carrier containing oleosin-TR peptide fusion protein;

FIG. 17 presents flow cytometry analysis bar graphs of tumor cells comprising different MOI values of the oil body carrier containing oleosin-Z_{Her2} peptide fusion protein;

FIG. 18 presents flow cytometry analysis bar graphs of tumor cells comprising different MOI values of the oil body carrier containing caleosin-Z_{Her2} peptide fusion protein;

FIG. 19 presents flow cytometry analysis bar graphs of tumor cells comprising different MOI values of the oil body carrier containing oleosin-TR peptide fusion protein;

FIG. 20 presents fluorescence microscope images of MCF7/HER18 cells comprising the oil body carrier containing oleosin-Z_{Her2} peptide fusion protein at different time points;

FIG. 21 presents fluorescence microscope images of SKOV3 cells comprising the oil body carrier containing oleosin-Z_{Her2} peptide fusion protein at different time points;

FIG. 22 presents fluorescence microscope images of MCF7/Her18 cells comprising the oil body carrier containing caleosin-Z_{Her2} peptide fusion protein at different time points;

FIG. 23 presents fluorescence microscope images of SKOV3 cells comprising the oil body carrier containing caleosin-Z_{Her2} peptide fusion protein at different time points;

FIG. 24 presents fluorescence microscope images of MCF7 cells comprising the oil body carrier containing oleosin-TR peptide fusion protein at different time points;

FIG. 25 presents fluorescence microscope images of SKOV3 cells comprising the oil body carrier containing oleosin-TR peptide fusion protein at different time points;

FIG. 26 presents flow cytometry analysis bar graphs of tumor cells comprising the oil body carrier containing oleosin-Z_{Her2} peptide fusion protein at different time points;

FIG. 27 presents flow cytometry analysis bar graphs of tumor cells comprising the oil body carrier containing caleosin-Z_{Her2} peptide fusion protein at different time points;

FIG. 28 presents flow cytometry analysis bar graphs of tumor cells comprising the oil body carrier containing oleosin-TR peptide fusion protein at different time points;

FIG. 29 presents confocal microscope images of the oil body carrier of the present invention after reacting with SKOV3 cells;

FIG. 30 presents bar graphs showing the influence of the oil body carriers or compositions covering lycopene of the present invention on cell viability;

FIG. 31 presents bar graphs showing the influence of the oil body carriers or compositions covering curcumin of the present invention on cell viability;

FIG. 32 presents curve diagrams showing the influence of the compositions covering lycopene of the present invention on cell viability;

FIG. 33 presents curve diagrams showing the influence of the compositions covering curcumin of the present invention on cell viability;

FIG. 34A presents molecular image analysis pictures showing the distribution of the oil body carrier of the present invention in the mouse body;

FIG. 34B is a bar graph showing the distribution of the oil body carrier of present invention in the mouse body;

FIG. 34C presents images of tissue slices showing the distribution of the oil body carrier of present invention in the mouse body; and

FIG. 35 presents fluorescence microscope images of the oil body carriers of the present invention after reacting with tumor tissue slices.

### DESCRIPTION OF THE PREFERRED EMBODIMENT

Unless there is an explanation in this article otherwise, the words “a”, “an”, “the”, and other analogous words in this specification (especially in the following claims) should be considered interchangeably as a singular or plural form.

It has been discovered that the oil body in a plant seed has a large surface area, and thus can be used to cover or embed a large amount of signal molecules to serve as a “real-time” signal amplifier moving in the body of an organism to tract and confirm the origin of a disease, or can be used to cover a fat-soluble medicament to serve as a drug delivery system. The present invention utilizes the above features and combines techniques in molecular biology to provide an artificial oil body carrier (which is called “oil body carrier” hereinafter) through a simple preparation procedure.

The oil body carrier of the present invention comprises a fusion protein and a lipid, wherein the fusion protein comprises an oil body protein and a ligand peptide, an antibody peptide, a cell penetrating peptide, or combinations thereof.

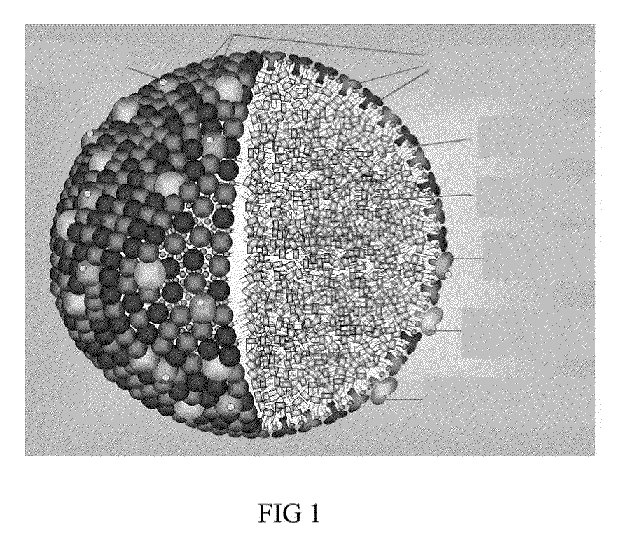

As shown in FIG. 1, the structure of a plant oil body is a spherical molecule mainly consisting of neutral fats, and the spherical molecule is surrounded by a layer of phospholipid (PL), which is like a cell membrane, and nearly the whole surface of the phospholipid layer embed s oil body proteins, including oleosin (as a structural protein) and minor caleosin and steroleosin. The major components in an oil body are neutral fats (mainly triacylglycerol, TAG) and a little phospholipid and oil body proteins (which can be seen in Chen et al., 1998, Identification of three novel unique proteins in seed oil bodies of sesame. Plant Cell Physiol. 39: 935-941, which is entirely incorporated hereinto by reference).

There is no limit for the oil body protein comprised in the fusion protein of the oil body carrier of the present invention. Preferably, the oil body protein is from the seeds of a plant, such as a plant selected from a group consisting of sesame, olive, soybean, peanut, sunflower, mustard flower, flax, safflower, and combinations thereof. More preferably, the oil body protein is from the seeds of sesame. In one embodiment of the present invention, oleosin from sesame seeds is used to construct a fusion protein, and the oleosin comprises an amino acid sequence of SEQ ID NO: 1; alternatively, caleosin from sesame seeds is used to construct a fusion protein, and the caleosin comprises an amino acid sequence of SEQ ID NO: 2.

For a certain plant seed, an average particle size of an oil body carrier obtained by using caleosin is usually smaller than that by using oleosin. For instance, when oleosin of sesame seeds is used, the average particle size of an obtained oil body carrier is about 500 to 2,000 nm, whereas when caleosin of sesame seeds is used, the average particle size of an obtained oil body carrier is about 50 to 200 nm.

The fusion protein in the oil body carrier of the present invention further comprises a ligand peptide and/or an antibody peptide to make the oil body carrier become a delivery carrier with specificity (i.e., a targeting carrier), thereby providing an “active targeting drug delivery system.” Because a ligand peptide or antibody peptide can recognize receptors on the surface of cancer cells precisely, an oil body carrier covering an anti-cancer drug can be delivered to a cancer area or acts on cancer cells directly to increase the local drug concentration without affecting normal cells through the combination of the receptors with the ligand peptide or antibody peptide. In addition, through the above mechanism, the phagocytosis and fusion activity of cancer cells toward an oil body carrier can be stimulated to engulf an anti-cancer drug into the cancer cells to threat and prevent drug-resistance.

There is no limit for the ligand peptide or antibody peptide comprised in the fusion protein of the present invention, as long as the ligand peptide or antibody peptide can provide the function of specifically recognizing particular cells. For example, in the treatment of breast cancer, a trastuzumab (commercial name: Herceptin) antibody peptide can be used.

As for the ligand peptide, for instance, in the treatment of breast cancer or ovarian cancer, a ligand peptide of HER2/neu protein receptors or α₅β₃ integrin receptors can be adopted. It is known that HER2/neu protein receptors, belonging to the epidermal growth factor receptor (EGFR), are present on the surface of many different cancer cells and play an important role in the carcinogenic mechanism, and thus can serve as specific biomarkers on the surface of cancer cells. Nord et al. developed a Z_{Her2} peptide which can bond to HER2/neu protein receptors specifically to serve as a ligand for the receptors (see Nord et al., Binding proteins selected from combinatorial libraries of an α-helical bacterial receptor domain., Nat Biotechnol., 1997, 15: 772-777, which is entirely incorporated hereinto by reference). Z_{Her2} peptide comprises 58 amino acids, the amino acid sequence of which is shown as Sequence ID NO:3, and the molecular weight thereof (about 7 to 15 kilodaltons) is much smaller than that of a monoclonal antibody (about 150 kilodaltons), and thus Z_{Her2} peptide can easily penetrate a cell membrane. In addition, α₅β₃ integrin receptors on cell surface also involve in the carcinogenic mechanism (see Giuffrida et al., Int J Oncol., Modulation of integrin expression on mesotheliomas: the role of different histotypes in invasiveness., 1999, 15(3):437-42, which is entirely incorporated hereinto by reference), and thus a ligand peptide (which is called “RGD peptide” hereinafter) of the α₅β₃ integrin receptors can be used to construct a fusion protein in the present invention.

In an embodiment of the present invention, Z_{Her2} peptide or RGD peptide along with the oleosin (comprising an amino acid sequence of SEQ ID NO: 1) or caleosin (comprising an amino acid sequence of SEQ ID NO: 2) of the seeds of sesame is used to construct a fusion protein, wherein RGD peptide comprises an amino acid sequence of SEQ ID NO: 4.

Cell penetrating peptides usually may bring many kinds of substances, penetrate directly through a cell membrane, and enter into a cell in a non-receptor-dependent way, and thus can assist or promote an oil body carrier to enter into target cells. Therefore, in the fusion protein of the present invention, a ligand peptide or antibody peptide may be also combined with a cell penetrating peptide to further elevate the cell penetrating efficiency of an oil body carrier.

The fusion protein of the present invention may comprise any known cell penetrating peptides, for example, TAT (an activator of transcription of human immunodeficiency virus 1, HIV-1), VP22 (a protein from Herpes simplex virus 1, HSV-1), Antp (a fruit fly antennapedia transcription homeoprotein), etc (see Fawell et al., Tat-mediated delivery of heterologous proteins into cells., Proc Natl Acad Sci USA., 1994, 18; 91(2): 664-668; Elliott et al., Intercellular trafficking and protein delivery by a herpesvirus structural protein., Cell., 1997, 24;88(2):223-233; and Derossi et al., The third helix of the Antennapedia homeodomain translocates through biological membranes., J Biol Chem., 1994, 8; 269 (14): 10444-10450, which are entirely incorporated hereinto by reference). In one embodiment of the present invention, an RGD peptide and a TAT peptide comprising an amino acid sequence of SEQ ID NO:5 are used to construct a fusion protein.

Accordingly, the oil body carrier of the present invention also can serve as a passive targeting drug delivery system; that is, a drug delivery system without receptor-dependency and specificity. By combining an oil body protein and the aforesaid cell penetrating peptides to provide a fusion protein, and utilizing the property of the cell penetrating peptides (i.e., bringing many kinds of substances and penetrating through a cell membrane directly and entering into a cell with a non-receptor-dependent way), an oil body carrier can enter into cells without via receptors to achieve a passive drug delivery effect.

There is no limit for the lipid comprised in the oil body carrier of the present invention. For instance, the lipid can be selected from a group consisting of triacylglycerol, olive oil, sesame oil, soybean oil, peanut oil, mineral oil, flax oil, safflower oil, and combinations thereof. The lipid is preferably triacylglycerol, sesame oil, or a combination thereof. More preferably, the lipid is sesame oil.

Through the adjustment of the proportion of fusion protein and lipid, oil body carriers with different average particle size can be prepared. Generally, the average particle size of an oil body carrier is inversely proportional to the proportion of fusion protein and lipid (i.e., fusion protein/lipid); the lesser the lipid, the smaller the average particle size of an oil body carrier. In the oil body carrier of the present invention, the weight/volume (μg/μl) ratio of fusion protein and lipid is usually at least about 1/25, and preferably is at least about 1/1, and more preferably is about 2/1 to about 30/1.

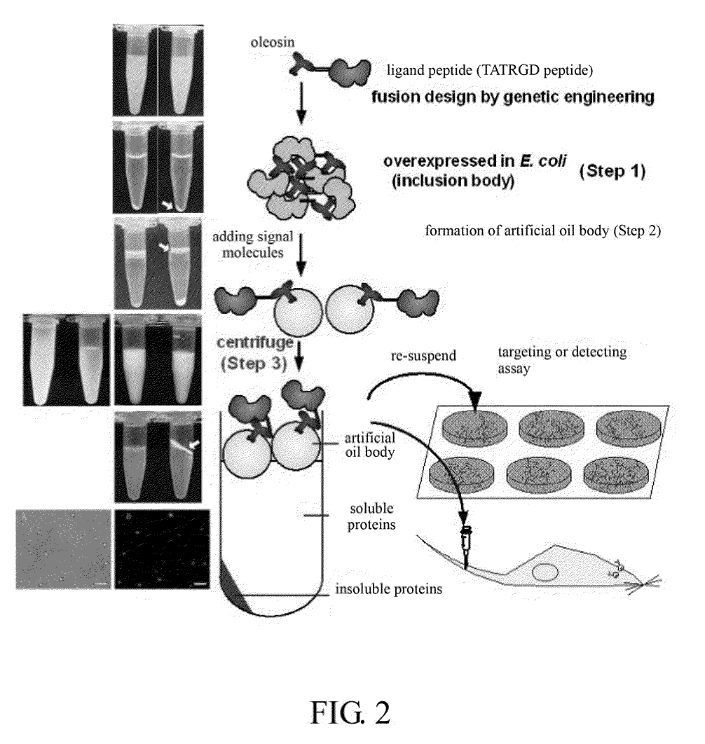

As shown in FIG. 2, the oil body carrier of the present invention can be prepared by the following method (but not limited thereby). First, a nucleic acid molecule of an oil body protein is combined with a nucleic acid molecule of a ligand peptide, an antibody peptide, a cell penetrating peptide, or combinations thereof in an expression vector using a gene recombination technique, and the expression vector is transformed into a host cell (e.g., Escherichia coli) to conduct the expression to prepare a fusion protein comprising the oil body protein and the ligand peptide, the antibody peptide, or the cell penetrating peptide. Then, the fusion protein and a lipid are mixed in a buffer solution, and the mixture is vibrated with an ultrasonic equipment to prepare the oil body carrier of the present invention.

It was discovered that, during the above preparation process, the pH value of the buffer solution may influence the average particle size and stability of the resultant oil body carrier. Generally, the pH value of a buffer solution is preferably at least about 7.0, and more preferably is about 7.0 to about 9.0.

In the better embodiment of the present invention, the following conditions are adopted to prepare an oil body carrier: (1) using oleosin of sesame seeds, an RGD peptide, and a TAT peptide to construct a fusion protein: (2) using olive oil as lipid; (3) the weight/volume (μg/μl) ratio of the fusion protein and the lipid is about 20/1; and (4) the pH value of a buffer solution is about 7.5. With the above conditions, an oil body carrier with an average particle size of about 20 to about 60 nm can be prepared.

Compared to the prior art, the present invention may control the particle size of an oil body carrier more conveniently, and thus can provide an oil body carrier suitable for any dosage form. In addition, the average particle size of the oil body carrier of the present invention can reach several tens of nanometers to sub-microns, and thus it can be absorbed by the human body more easily. In the preparation of an oil body carrier with an injection dosage form, the average particle size thereof can be controlled to range from about 10 nm to about 300 nm.

The present invention also provides a composition for target therapy and/or detection with excellent delivery property, which comprises the oil body carrier of the present invention and a medicament, a signal molecule, or a combination thereof, and the oil body carrier is defined as the above.

In addition to anti-cancer drugs, the composition of the present invention may also comprise any other medicaments, and preferably comprises fat-soluble medicaments. For example, the composition of the present invention may comprise a medicament selected from a group consisting of lycopene, curcumin, camptothecin, fat-soluble antibiotics, cucurbitacin, vinorelbine (commercial name: Navelbin), and combinations thereof.

The composition of the present invention may also comprise any known signal molecules to achieve a desired detection purpose. For instance, the signal molecule may be selected from a group consisting of cadmium-cesium quantum dots, fluorescein isothiocyanate (FITC), Alizarine Yellow R (5-[(p-nitrophenyl)azo]salicylic acid sodium salt), Nile Red (9-diethylamino-5H-benzo[α]phenoxazine-5-one), and combinations thereof. In light of the application of cadmium-cesium quantum dots, because when cadmium-cesium quantum dots with different size are excited with light having different wavelengths, the quantum dots emit florescence with different wavelengths and such a property can be used to prepare an oil body carrier that emits florescence with different colors. Herein, when a ligand peptide or an antibody peptide is used to construct a fusion protein comprised in the composition of the present invention, the composition of the present invention may have a target detection function to mark or tag the position of cancer cells or disease origins.

As shown in the following examples, the composition of the present invention may mark cancer cells efficiently, and thus can be applied in the real-time in vivo image observation for cancer. The composition may also deliver drugs precisely, and thus can accurately kill cancer cells to lower the side effect of killing normal cells. Therefore, the composition of the present invention has the effects of real-time monitoring and treatment.

Given the above, in terms of carrying signal molecules, covering medicaments, and modifying or altering targeting peptides, compared with the conventional carriers, the oil body carrier of the present invention has simple operability and better carrier property, and thus can be broadly applied in industries such as western medicine, medicine inspection, biomedicine materials, animal vaccines, biotechnology, etc.

The present invention will be further illustrated in details with specific examples as follows. After referring to the examples described in the following paragraphs, people skilled in this field can easily appreciate the basic spirit, other invention purposes of the present invention, technical methods adopted in the present invention and better embodiments. However, the following examples are provided only for illustrating the present invention, and the scope of the present invention is not limited thereby.

### EXAMPLE 1

### Preparation of Oil Body Carriers

The oil body carrier of the present invention was prepared according to the flowchart shown in FIG. 2.

Step 1. Construction of Expression Vectors

The following three nucleic acid molecules were constructed in expression vectors by using gene recombination techniques.

(1) Nucleic Acid Molecule of Oleosin (N-Terminal)-Z_{Her2} Peptide (C-Terminal) Fusion Protein

A linker (comprising an amino acid sequence of SEQ ID NO:7) gene comprising a nucleic acid sequence of SEQ ID NO:6 was used to combine the oleosin gene (comprising a nucleic acid sequence of SEQ ID NO: 8) from sesame seeds and the gene (comprising a nucleic acid sequence of SEQ ID NO: 9) of a ligand peptide (i.e., Z_{Her2} peptide) of HER2/neu protein. The detailed procedures are as follows. First, a pET-Z_{Her2} vector was purified to serve as a template DNA, and primers were used to obtain a Z_{Her2} gene fragment (507 base pairs) via polymerase chain reaction (PCR). Then, Z_{Her2} gene was incised with Nco I and Hind III restriction enzymes, and was linked to a pBluescript II (SK+) vector, and the resultant recombinant vector was transformed into E. coli. DH5α host cells. The host cells were incubated in a solid LB (Luria-Bertani) medium containing ampicillin and X-gal (purchased from Sigma Co.), and screening was conducted, in which white colonies were picked to obtain a transformant comprising a pBluescript II-Z_{Her2} recombinant vector. Finally, a Z_{Her2} gene fragment in the recombinant vector was incised with Nco I and Hind III restriction enzymes, and was linked to a recombinant vector comprising a pJo1-oleosin gene to obtain an expression vector comprising pJo1-oleosin gene-Z_{Her2}.

(2) Nucleic Acid Molecule of Caleosin (N-Terminal)-Z_{Her2} Peptide (C-Terminal) Fusion Protein

A linker (comprising an amino acid sequence of SEQ ID NO:11) gene comprising a nucleic acid sequence of SEQ ID NO:10 was used to combine the caleosin gene (comprising a nucleic acid sequence of SEQ ID NO: 12) from sesame seeds and the gene of Z_{Her2} peptide (comprising a nucleic acid sequence of SEQ ID NO: 9). The detailed procedures are as follows. First, a pET-caleosin gene vector was purified to serve as a template DNA, and primers were used to obtain a caleosin gene fragment (748 base pairs) via PCR. Then, the gene fragment was incised with an Nde I restriction enzyme, and was linked to a pET-29a (+) vector (purchased from Novagene Co.), and the resultant recombinant vector was transformed into E. coli. DH5α host cells. The host cells were incubated in a solid LB medium containing kanamycin (purchased from Sigma Co.), and screening was conducted to obtain a transformant comprising a pET-29a-caleosin gene recombinant vector. Finally, a Z_{Her2} gene fragment in the pBluescript II-Z_{Her2} recombinant vector on the above (1) was incised with Eco

RV and Hind III restriction enzymes, and was linked to a recombinant vector comprising pET-29a-caleosin gene to obtain an expression vector comprising pET-29a-caleosin gene-Z_{Her2}.

(3) Nucleic Acid Molecule of Oleosin (N-Terminal)-TATRGD (Which is Called “TR” Hereinafter) Peptide (C-Terminal) Fusion Protein

A linker (comprising an amino acid sequence of SEQ ID NO:14) gene comprising a nucleic acid sequence of SEQ ID NO: 13 was used to combine the oleosin gene (comprising a nucleic acid sequence of SEQ ID NO: 8) from sesame seeds and a gene (comprising a nucleic acid sequence of SEQ ID NO: 15) of a ligand peptide (i.e., RGD) of α₅β₃ integrin, and a gene (comprising a nucleic acid sequence of SEQ ID NO: 16) of a TAT peptide. The detailed procedures are as follows. First, several sets of overlapping primers were designed, and a TATRGD (TR) gene with 129 base pairs was synthesized by PCR. Then, the TATRGD gene was treated with a Dpn I restriction enzyme, and was linked to a pJo1-oleosin gene vector with a ligase to obtain an expression vector comprising pJo1-oleosin gene-TR gene, which further contains a T7 promoter to regulate transcription.

After being constructed, the above expression vectors were individually transformed into E. coli BL21 (DE3) host cells (purchased from Novagen Co.), and plasmids in the host cells were extracted to confirm nucleic acid sequences of the expression vectors.

The above method refers to Sambrook et al., The Condensed Protocols From Molecular Cloning: A Laboratory Manual 2006, which is entirely incorporated hereinto by reference.

Step 2. Expression of Fusion Proteins

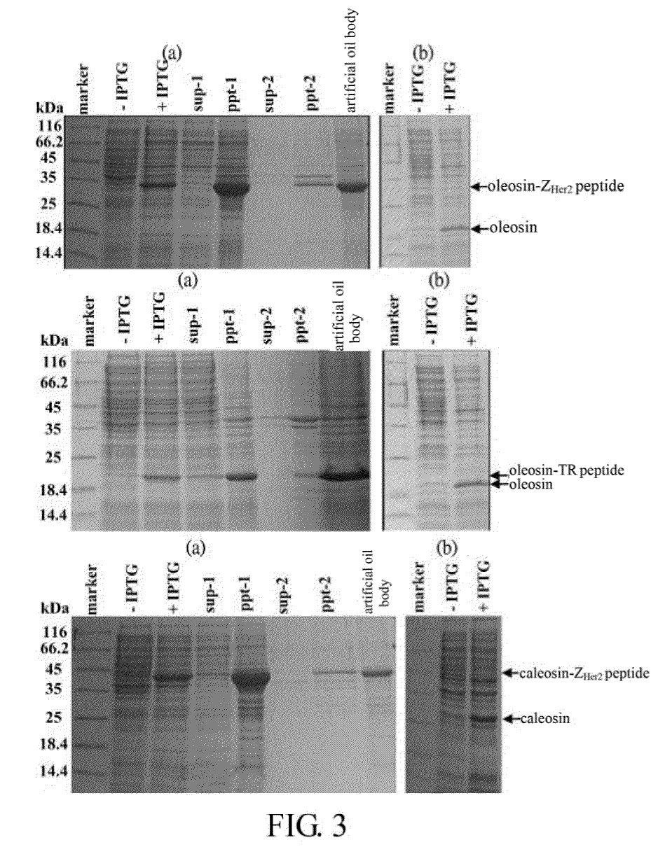

The host cells obtained in Step 1 were induced with 0.05 mM IPTG (isopropyl-β-D-1-thiogalactopyranoside, purchased from USB Co.) to overexpress the fusion proteins, and the broth was collected. The broth was centrifuged under 6,500 rpm for 10 minutes, and the deposited host cells were suspended with a TE buffer (Tris-EDTA buffer, purchased from Sigma Co.) solution of about 1/10 broth volume, and were added into an SDS-PAGE 4X sample buffer (purchased from Sigma Co.) and thoroughly mixed. The mixture was heated under 95° C. for about 10 minutes, and a protein electrophoresis analysis was conducted. The results are shown in FIG. 3.

Step 3. Addition of Lipid and Signal Molecules

The fusion protein (50 mg) prepared in Step 2 were added into a tube, and 50 mg triacylglycerol, 150 mg phospholipid, and 2.5 μg florescence dye (Alizarine Yellow, purchased from Widetex Biotech, Co., LTD., Taiwan) or cadmium-cesium quantum dots (provided by Prof Rong Fuh Louh, Feng Chia University) was added thereinto. The mixture was vibrated every 5 minutes for five times (10 seconds, amplitude: 20, pulser: 0.5) with an ultrasonic equipment (mode: Sonics VCX130) to conduct the recombination of oil body. After the recombination, oil body carriers comprising three different fusion proteins (i.e., oleosin-Z_{Her2} peptide, caleosin-Z_{Her2} peptide, and oleosin-TR peptide) individually were prepared, and the carriers were observed with a fluorescence microscope.

Common biochemical analysis methods can be used to determine the content of three essential components (i.e., triacylglycerol, proteins, and phospholipid) in the oil body carriers. Herein, the triacylglycerol content was determined by examining and calculating the amount of ester bonds, the protein content was measured by a BCA protein assay (BioRad, Co.), and the phospholipid content was obtained by measuring the amount of inorganic phosphate.

### EXAMPLE 2

### Influence of the Ratio of Lipid and Fusion Proteins

A sodium phosphate buffer solution (950 μl, 0.01M, pH 7.5) and olive oil (50 μl) were added to 100 μg fusion proteins (i.e., oleosin-Z_{Her2} peptide, caleosin-Z_{Her2} peptide, or oleosin-TR peptide), and 150 μg phospholipid was added thereinto to obtain a mixture in which the weight/volume (μg/μl) ratio of fusion protein/lipid (i.e., olive oil) was 2/1. Then, the mixture was placed on ice and vibrated with ultrasonic for three times (efficiency: 15%, time: 20 seconds; run: 0.5 second, rest: 0.5 second) to obtain a desired oil body carrier. The above operation was repeated, and 400 μg, 200 μg, 100 μg, or 100 μg of fusion proteins and corresponding 20 μl, 20 μl, 100 μl, or 500 μl of olive oil were used to obtain an oil body carrier in which the weight/volume (μg/μl) ratio of fusion protein/lipid (i.e., olive oil) was 20/1, 10/1, 1/1, or 1/5.

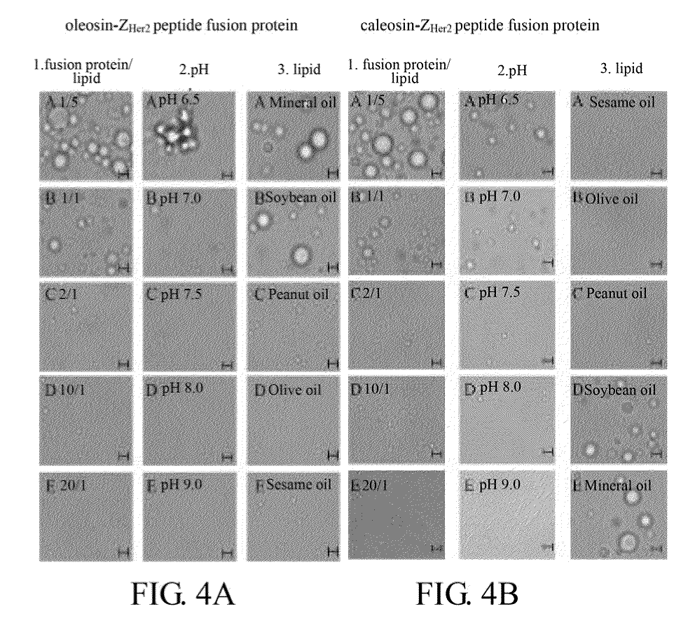

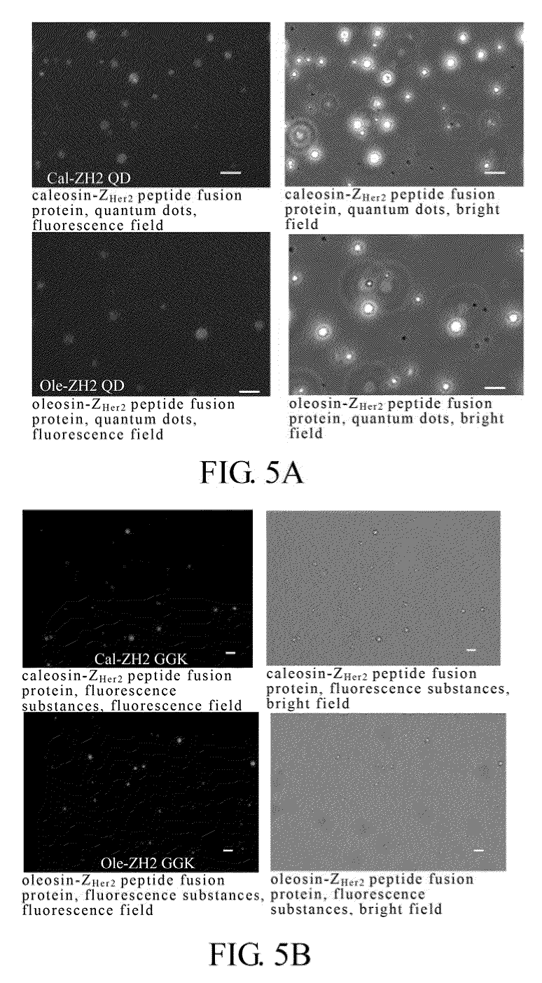

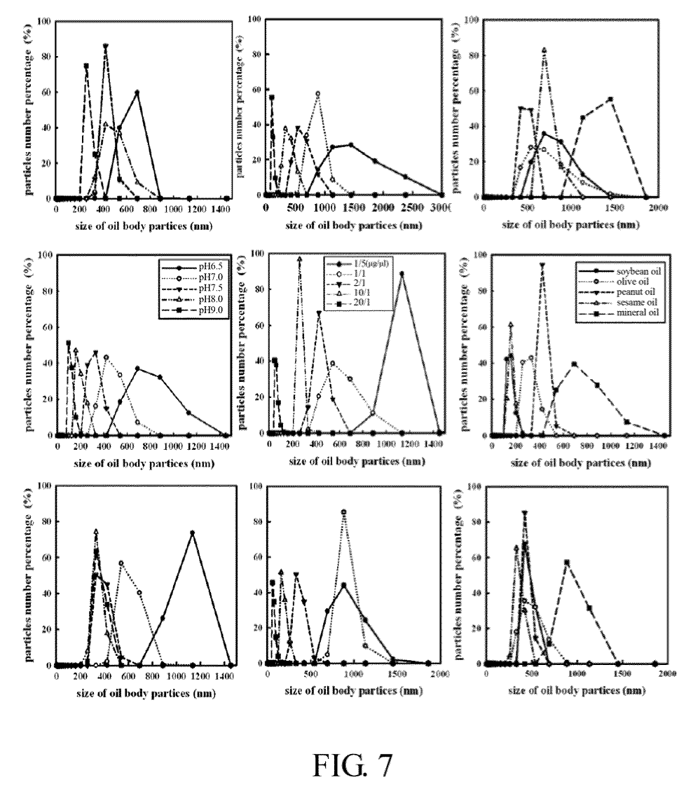

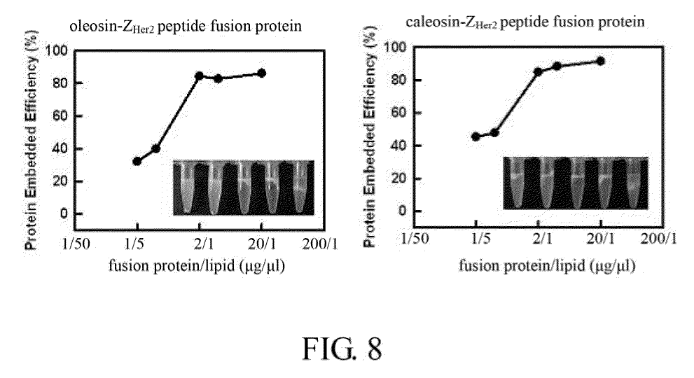

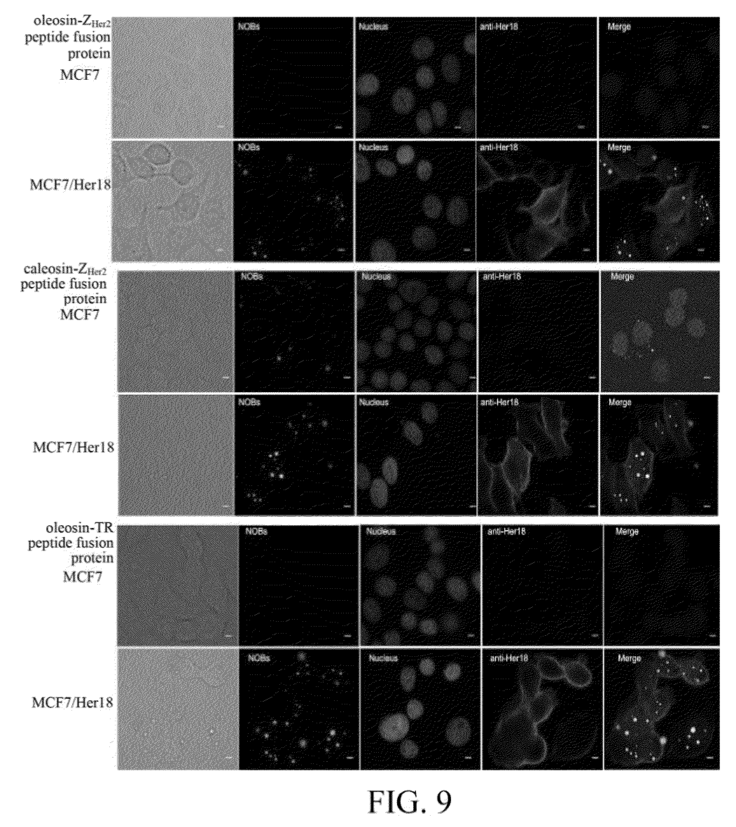

The conformation and turbidity of the oil body carriers were observed with a Nikon 104 optical microscope, which are shown in FIGS. 4A to 5B (conformation) and 6 (turbidity), respectively. The turbidity of the oil body carriers was calculated as described in following Example 5, and the particle size of the oil body carriers was analyzed with a particle size analysis instrument (Beckman Coulter, N4 Plus), wherein the ion strength was set at 0.1, and the particle size and distribution thereof were analyzed by using dynamic light scanning (DLS, argon laser beam: 633 nm, scanning angle: 90°, assay method: Contin) under 25° C. The results are shown in FIG. 7 and columns (a) in Tables 1 to 3. The embedded rate of fusion proteins was analyzed with SDS-PAGE, and the results are shown in FIG. 8.

### EXAMPLE 3

### Influence of pH Value

The fusion protein (100 μg oleosin-Z_{Her2} peptide, 90 μg caleosin-Z_{Her2} peptide, or 100 μg oleosin-TR peptide) was added into 950 μl sodium phosphate buffer solution (0.01M) with different pH values (pH 6.5, pH 7.0, pH 7.5, pH 8.0, or pH 9.0), and 50 μl olive oil was added thereinto. Then, the mixture was placed on ice and vibrated with ultrasonic for three times (efficiency: 15%, time: 20 seconds; run: 0.5 second, rest: 0.5 second) to prepare oil body carriers. The conformation and turbidity of the oil body carriers were observed with a Nikon 104 optical microscope, which are shown in FIGS. 4A to 5B (conformation) and 6 (turbidity), respectively, and the particle size of the oil body carriers was analyzed with a particle size analysis instrument, and the results are shown in FIG. 7 and columns (b) in Tables 1 to 3.

### EXAMPLE 4

### Influence of Lipid

The fusion protein (100 μg oleosin-Z_{Her2} peptide, 90 μg caleosin-Z_{Her2} peptide, or 100 μg oleosin-TR peptide) was added into 950 μl sodium phosphate buffer solution (0.01 M, pH 7.5), and 50 μl of different lipids (olive oil, sesame oil, soybean oil, peanut oil, or mineral oil) was individually added thereinto. Then, the mixture was placed on ice and vibrated with ultrasonic for three times (efficiency: 15%, time: 20 seconds; run: 0.5 second, rest: 0.5 second) to prepare oil body carriers. The conformation and turbidity of the oil body carriers were observed with a Nikon 104 optical microscope, which are shown in FIGS. 4A to 5B (conformation) and 6 (turbidity), respectively, and the particle size of the oil body carriers was analyzed with a particle size analysis instrument, and the results are shown in FIG. 7 and columns (c) in Tables 1 to 3.

TABLE 1
Particle size of the oil body carriers comprising oleosin-Z_{Her2} peptide
prepared (a) at different weight/volume ratios of fusion protein to
lipid in pH 7.5. (b) at different pH values (c) with various lipids

| (a) | | (b) | | (c) | |
|---|---|---|---|---|---|
| fusion protein/ | average | | average | | average |
| lipid (weight/ | particle | | particle | | particle |
| volume) ratio | size (nm) | pH | size (nm) | lipid | size (nm) |
| 1/5 | 1543.8 ± 95.8 | 6.5 | 896.5 ± 33.1 | mineral oil | 1264.1 ± 47.7 |
| 1/1 | 1113.8 ± 24.0 | 7.0 | 785.2 ± 30.4 | Peanut oil | 893.5 ± 14.1 |
| 2/1 | 786.2 ± 16.4 | 7.5 | 765.8 ± 30.7 | olive oil | 817.9 ± 14.7 |
| 10/1 | 558.9 ± 37.0 | 8.0 | 454.6 ± 21.0 | sesame oil | 745.1 ± 39.5 |
| 20/1 | 286.1 ± 22.2 | 9.0 | 318.3 ± 46.3 | soybean oil | 503.0 ± 23.3 |

TABLE 2
Particle size of the oil body carriers comprising caleosin-Z_{Her2} peptide
prepared (a) at different weight/volume ratios of fusion protein to lipid
in pH 7.5. (b) at different pH values (c) with various lipids

| (a) | | (b) | | (c) | |
|---|---|---|---|---|---|
| fusion protein/ | average | | average | | average |
| lipid (weight/ | particle | | particle | | particle |
| volume) ratio | size (nm) | pH | size (nm) | lipid | size (nm) |
| 1/5 | 1273.6 ± 48.5 | 6.5 | 634.0 ± 24.1 | mineral oil | 836.7 ± 12.7 |
| 1/1 | 843.2 ± 53.07 | 7.0 | 693.5 ± 30.1 | soybean oil | 463.1 ± 8.2 |
| 2/1 | 499.8 ± 12.9 | 7.5 | 538.1 ± 32.6 | peanut oil | 430.1 ± 7.7 |
| 10/1 | 163.9 ± 26.2 | 8.0 | 483.9 ± 48.0 | olive oil | 466.6 ± 64.4 |
| 20/1 | 87.4 ± 51.2 | 9.0 | 372.0 ± 59.5 | sesame oil | 431.5 ± 67.4 |

TABLE 3
Particle size of the oil body carriers comprising oleosin-TR peptide
prepared (a) at different weight/volume ratios of fusion protein to
lipid in pH 7.5. (b) at different pH values (c) with various lipids.

| (a) | | (b) | | (c) | |
|---|---|---|---|---|---|
| fusion protein/ | average | | average | | average |
| lipid (weight/ | particle | | particle | | particle |
| volume) ratio | size (nm) | pH | size (nm) | lipid | size (nm) |
| 1/5 | 1173.1 ± 24.1 | 6.5 | 787.7 ± 36.6 | mineral oil | 686.7 ± 66.7 |
| 1/1 | 783.1 ± 16.8 | 7.0 | 472.5 ± 24.3 | soybean oil | 139.6 ± 14.5 |
| 2/1 | 412.5 ± 23.4 | 7.5 | 339.3 ± 72.0 | peanut oil | 397.4 ± 48.4 |
| 10/1 | 200.9 ± 47.7 | 8.0 | 159.6 ± 25.8 | olive oil | 328.2 ± 34.7 |
| 20/1 | 42.9 ± 24.3 | 9.0 | 77.9 ± 3.3 | sesame oil | 177.1 ± 63.6 |

As shown in Tables 1 to 3, FIGS. 4A, 4B, and 7, the average particle size of the prepared oil body carriers ranged from 10 to 2,000 nm, and became smaller with the decrease of the lipid/fusion protein ratio. Therefore, oil body carriers with different average particle size can be prepared by adjusting the lipid/fusion protein ratio. Besides, the average particle size of the oil body carriers was relatively small in an alkalic environment. Furthermore, FIG. 8 shows that when the weight/volume (μg/μl) ratio of fusion proteins and lipid was about 2/1, the embedded rate of the fusion proteins reached its maximum.

### EXAMPLE 5

### Determination of Stability of Oil Body Carriers

The stability of the oil body carriers was determined with the following three methods.

A. Observation of Repulsion Force of Negative Charges

Through the observation of the repulsion force of negative charges or the steric hindrance effect caused by the protein coverage on the surface of oil body carriers, the stability of the oil body carriers can be determined, and the oil body gathering caused by the gradual disappearance of the repulsion force of negative charges can be observed by lowering the pH value of a solution. Herein, the oil body carrier was placed in a phosphate buffer solution with different pH values, and was placed still at room temperature for 12 hours, and the change of the oil body carrier was observed with an optical microscope. As can be seen in FIG. 2, after 12 hours under pH 7.5, the oil body carrier comprising oleosin or caleosin remained intact.

B. Measurement of Turbidity

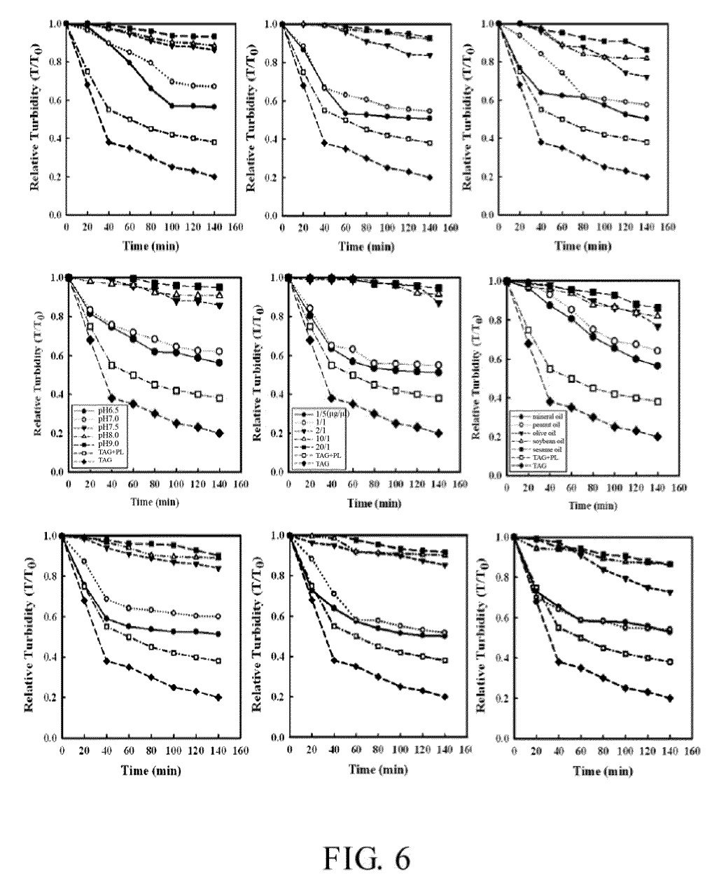

If an oil body carrier is intact, it stays in a suspended state in water, because the surface of the oil body carrier is hydrophilic and the carrier is highly soluble in water. On the contrary, if an oil body carrier is not intact, it floats on the water surface, because proteins on the surface of the oil body carrier cannot fold correctly, leading to the gathering of oil body carriers. Therefore, through the determination of the turbidity at the bottom of a solution, the intactness of an oil body carrier can be examined indirectly. Herein, 1 ml of the oil body carrier was placed in a disposable measuring tube, and the tube was sealed, and any vibration was prevented. The tube was placed still at room temperature for 140 minutes, and the turbidity in the tube was measured with a wavelength of 600 nm, wherein the relative turbidity is represented as T/T₀=10_{A}/10_{A0}=10^{A}/10^{2.0}, and A0 is 2.0. As can be seen in FIG. 6, after 140 minutes, the oil body carriers remained intact, indicating that the oil body carriers of the present invention have excellent stability.

C. Measurement of Zeta Potential

The oil body carriers were distributed in different environments (i.e., different weight/volume ratios of lipid and fusion protein, pH values, or lipids), and the variation of zeta potential on the surface of the oil body carriers was measured with a surface potential analysis instrument (Zetasizer Nano, Malvern #ZS90). The results are shown in Tables 4 to 6.

TABLE 4
Zeta potential of the oil body carriers comprising oleosin-Z_{Her2} peptide
prepared (a) at different weight/volume ratios of fusion protein to lipid
in pH 7.5. (b) at different pH values (c) with various lipids

| (a) | | (b) | | (c) | |
|---|---|---|---|---|---|
| fusion protein/ | zeta- | | zeta- | | zeta- |
| lipid (weight/ | potential | | potential | | potential |
| volume) ratio | (mV) | pH | (mV) | lipid | (mV) |
| 1/5 | −65.9 | 6.5 | −45.5 | mineral oil | −45.9 |
| 1/1 | −46.3 | 7.0 | −41.4 | soybean oil | −45.5 |
| 2/1 | −45.7 | 7.5 | −41.0 | peanut oil | −47.8 |
| 10/1 | −44.9 | 8.0 | −45.2 | olive oil | −47.5 |
| 20/1 | −41.6 | 9.0 | −36.0 | sesame oil | −44.7 |

TABLE 5
Zeta potential of the oil body carriers comprising caleosin-Z_{Her2} peptide
prepared (a) at different weight/volume ratios of fusion protein to lipid
in pH 7.5. (b) at different pH values (c) with various lipids

| (a) | | (b) | | (c) | |
|---|---|---|---|---|---|
| fusion protein/ | zeta- | | zeta- | | zeta- |
| lipid (weight/ | potential | | potential | | potential |
| volume) ratio | (mV) | pH | (mV) | lipid | (mV) |
| 1/5 | −64.2 | 6.5 | −52.2 | mineral oil | −48.4 |
| 1/1 | −55.8 | 7.0 | −48.4 | soybean oil | −45.1 |
| 2/1 | −46.2 | 7.5 | −42.8 | peanut oil | −49.2 |
| 10/1 | −48.3 | 8.0 | −48.9 | olive oil | −46.2 |
| 20/1 | −45.9 | 9.0 | −42.2 | sesame oil | −45.4 |

TABLE 6
Zeta potential of the oil body carriers comprising oleosin-TR peptide
prepared (a) at different weight/volume ratios of fusion protein to lipid
in pH 7.5. (b) at different pH values (c) with various lipids.

| (a) | | (b) | | (c) | |
|---|---|---|---|---|---|
| fusion protein/ | zeta- | | zeta- | | zeta- |
| lipid (weight/ | potential | | potential | | potential |
| volume) ratio | (mV) | pH | (mV) | lipid | (mV) |
| 1/5 | −54.2 | 6.5 | −56.8 | mineral oil | −54.0 |
| 1/1 | −52.8 | 7.0 | −54.8 | soybean oil | −51.7 |
| 2/1 | −48.3 | 7.5 | −47.7 | peanut oil | −49.3 |
| 10/1 | −47.9 | 8.0 | −47.2 | olive oil | −48.9 |
| 20/1 | −46.5 | 9.0 | −45.3 | sesame oil | −45.4 |

As shown in Tables 4 to 6, the stability of the oil body carriers increased with the decrease of the lipid/fusion protein ratio, and the oil body carriers were relatively stable in an alkalic environment.

### EXAMPLE 6

### In Vitro Examination of Target Function of Oil Body Carriers-Fixed Tumor Cell Assay

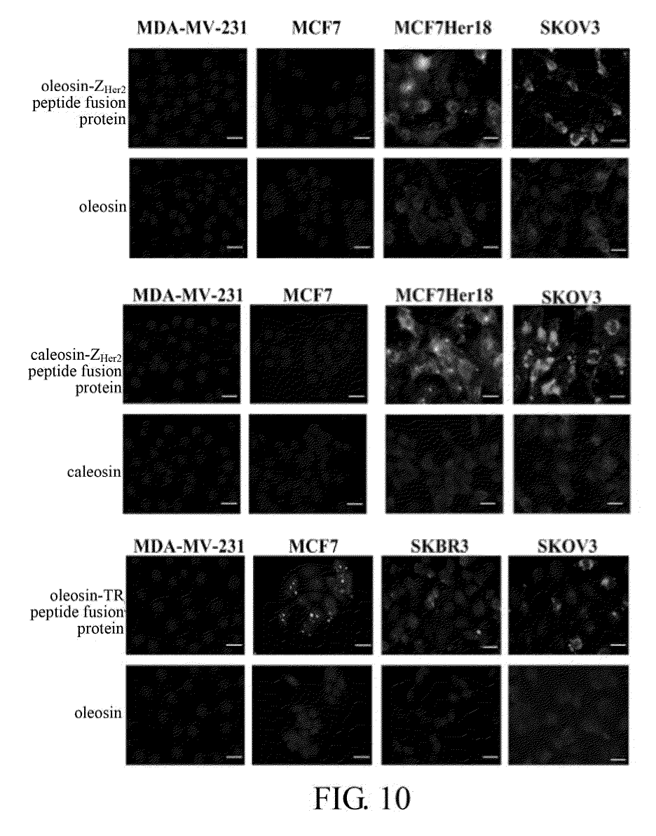

Human breast cancer cell lines, MCF7 cells and MCF7/Her18 cells (i.e., MCF7 cells with HER2/neu receptors on the surface thereof), were inoculated in a 24-wells plate, respectively. The next day, the cells were washed with a phosphate buffer solution (PBS, pH 7.4), and were fixed with 3.7 wt/vol % formaldehyde at room temperature for 20 minutes, and were washed again with pH 7.4 PBS. The oil body carrier (comprising 2.5 μg/ml-PBS oleosin-Z_{Her2} peptide) prepared in Example 1 was added to the fixed cells, and were reacted at 25° C. for an hour in pH 7.4 PBS. Then, the cells were washed with pH 7.4 PBS comprising 1/1000 Tween-20 (purchased from USB Co.) twice, and washed with pH 7.4 PBS once. A blocking solution (3 wt/vol % fetal bovine serum albumin dissolved in PBS) was then added to the cells, and reacted at room temperature for an hour, and a primary antibody, anti-HER2/neu (9G6, Santa Cruz Biotechnology Co., LTD., Santa Cruz, Calif., USA), was diluted in a proportion of 1:200 and added to the cells to react at room temperature for at least an hour. Thereafter, the cells were washed three times with PBS, and were reacted with anti-mouse IgG-TRIAC diluted in a proportion of 1:500 (Jackson ImmunoResearch Laboratories Co., West Grove, Pa., USA) for an hour, and were washed with PBS for three times. The nuclei of the cells were stained with 15,000-fold DAPI (diamidino-2-phenylindole) and washed, and the cells were observed under a fluorescence microscope (Olympus, IX71). The results are shown in FIG. 9.

As can be seen in FIG. 9, the oil body carriers targeting HER2/neu receptors (i.e., the oil body carrier comprising oleosin-Z_{Her2} peptide or caleosin-Z_{Her2} peptide fusion protein) specifically marked or tagged fixed MCF7/Her18 cells that had overexpressed HER2/neu receptors.

### EXAMPLE 7

### In Vitro Examination of Target Function of Oil Body Carriers-Live Tumor Cell Assay

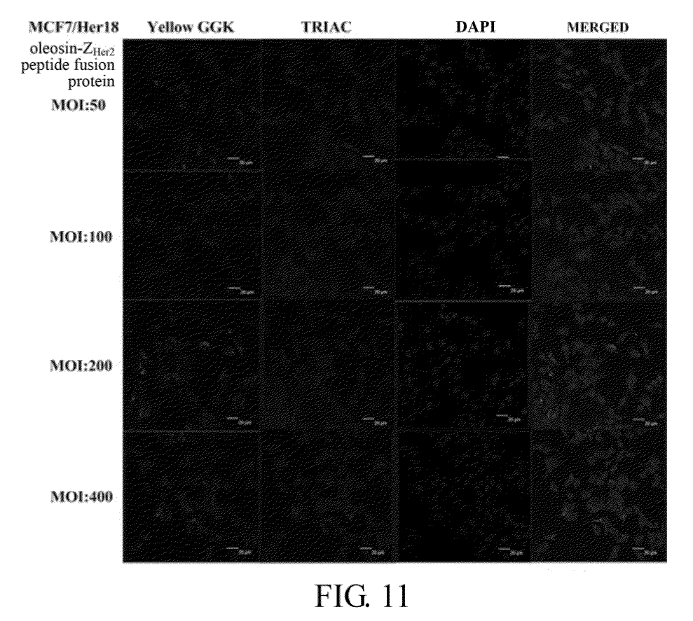

SKBR3 cells (breast cancer cells overexpressing HER2/neu receptors), MDA-MV-231 cells (a control group, breast cancer cells without overexpressing HER2/neu receptors), or 5×10⁵ cells (MCF7 cells, MAF7/Her18 cells, SKOV3 cells (ovarian cancer cells with overexpressing HER2/neu receptors)) were inoculated in a 24-wells plate individually, and were incubated in a cell incubator (containing 5 vol % carbon dioxide) at 37° C. for 24 hours. The next day, the cells were washed with a DMEM/F12 incubation solution (GIBCO Invitrogen Corporation, New York, USA), and the oil body carrier (comprising 0.025 μg/ml-PBS oleosin-Z_{Her2} peptide or caleosin-Z_{Her2} peptide) prepared in Example 1 was added into the washed cells, and was reacted in a DMEM/F12 incubation solution in an incubator (containing 5 vol % carbon dioxide) at 37° C. for 4 hours, and the cells were then washed with pH 7.4 PBS three times. Then, the cells were fixed with 2.5 wt/vol % formaldehyde at room temperature for 40 minutes, and were washed again with pH 7.4 PBS. A blocking solution (3 wt/vol % fetal bovine serum albumin dissolved in PBS) was then added to the cells, and reacted at room temperature for an hour, and a primary antibody, anti-HER2/neu (9G6, Santa Cruz Biotechnology Co., Santa Cruz, Calif., USA), was diluted in a proportion of 1:200 and added to the cells to react at room temperature for at least an hour. Thereafter, the cells were washed three times with PBS, and were reacted with anti-mouse IgG-TRIAC diluted in a proportion of 1:500 (Jackson ImmunoResearch Laboratories Co., West Grove, Pa., USA) for an hour, and were washed with PBS for three times. The nuclei of the cells were stained with 15,000-fold DAPI and washed, and the cells were observed with a fluorescence microscope. The results are shown in FIG. 10.

As can be seen in FIG. 10, the oil body carriers targeting HER2/neu receptors (i.e., the oil body carrier comprising oleosin-Z_{Her2} peptide or caleosin-Z_{Her2} peptide fusion protein) specifically marked or tagged live MCF7/Her18 cells and SKOV3 cells with overexpressing HER2/neu receptors. The oil body carrier comprising oleosin-TR peptide fusion protein had specificity toward MCF7 cells, SKOV3 cells, and MCF7/Her18 cells overexpressing α₅β₃ integrin.

### EXAMPLE 8

### Optimum Reaction Concentration of Oil Body Carriers and Tumor Cells

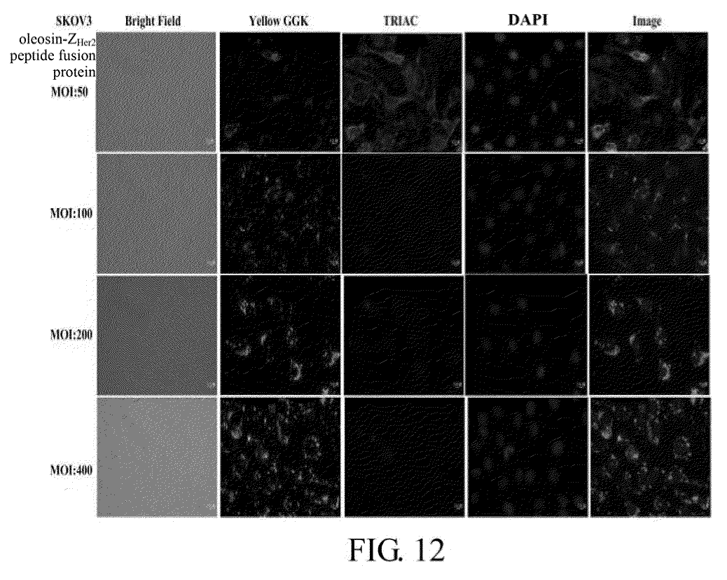

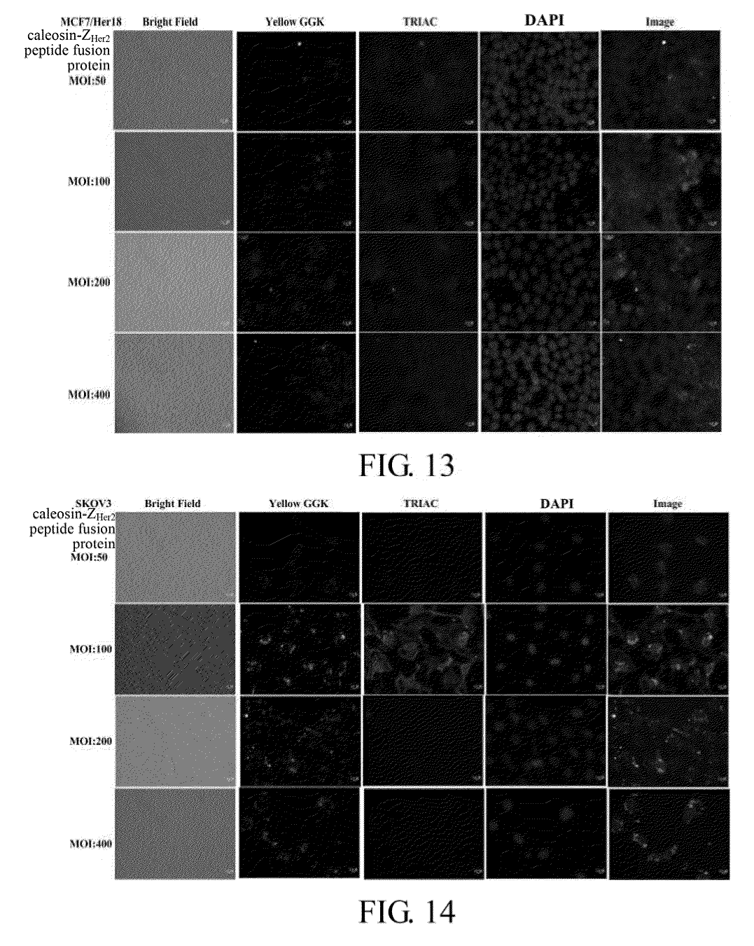

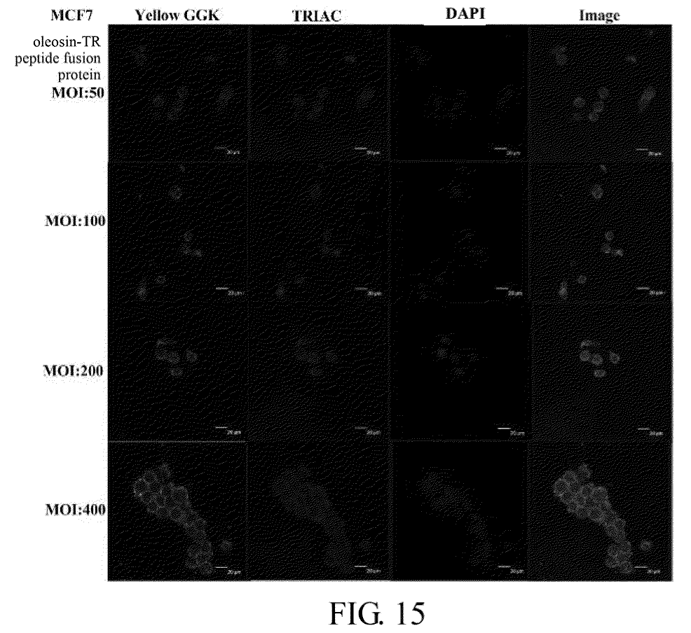

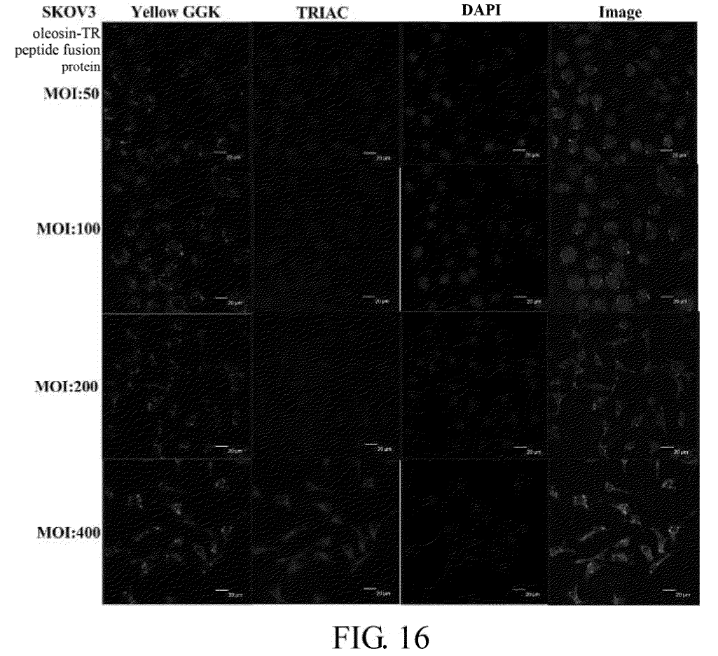

The different multiplicity of infection (MOI) values (MOI 100, MOI 200, or MOI 400) of the oil body carriers and tumor cells were used to determine the optimum reaction concentration thereof. Herein, the cell lines used were MCF7/Her18 cells with overexpressing HER2/neu receptors, MCF7 cells without overexpressing HER2/neu receptors, and SKOV3 ovarian cancer cells with overexpressing HER2/neu receptors. The cells were observed with a fluorescence microscope and a flow cytometry (BD FACSCanto (Argon-Ion Laser 488 nm, He-Ne Laser 633 nm)). The MOI value is defined as a ratio of the number of oil body carriers to that of tumor cells, which may be transferred as a concentration unit according to the following formulas, and the results are shown in FIGS. 11 to 16:

MOI 100=1.25×10⁻² μg/μl

MOI 200=2.5×10⁻² μg/μl

MOI 400=5×10⁻² μg/μl.

As shown in FIGS. 11 to 16, through the observation under the fluorescence microscope, it was found that the oil body carriers comprising Z_{Her2} peptide specifically recognized the cells with overexpressing HER2/neu receptors, and the number of oil body carriers entering into the cells elevated with the increase of the MOI value. The number reached its maximum when the MOI value was 200.

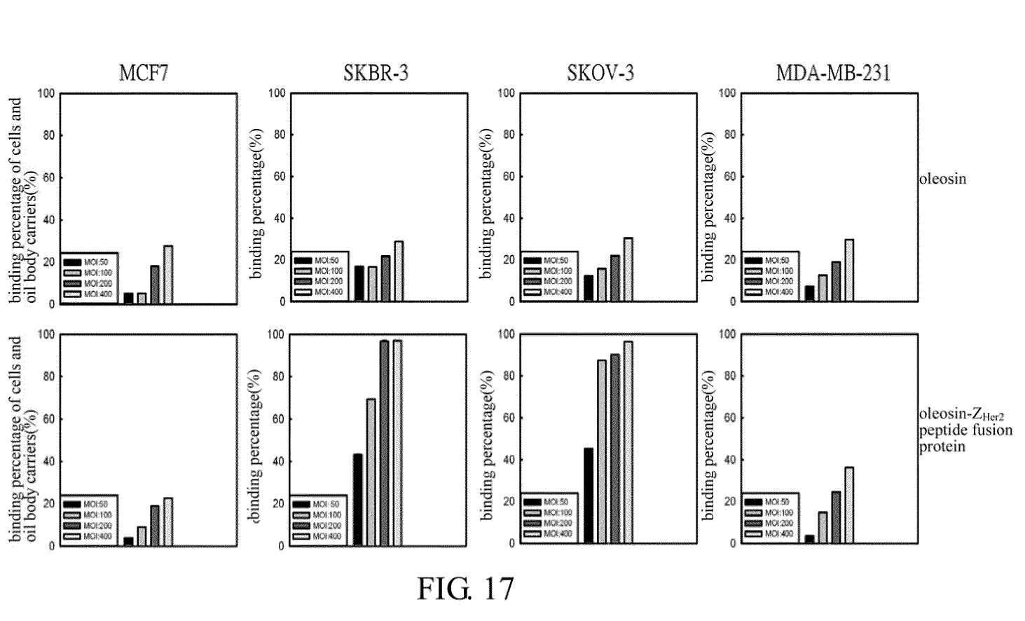

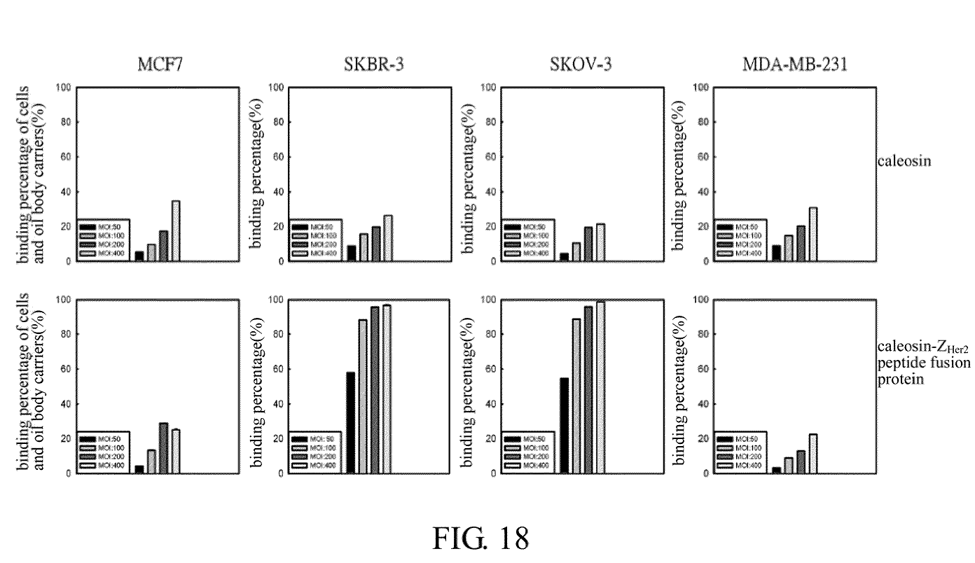

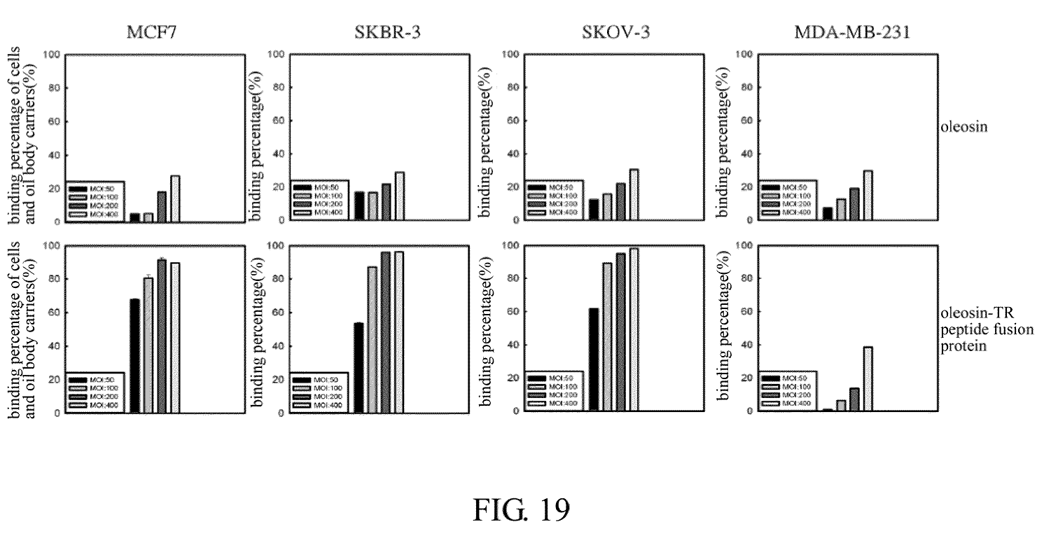

In addition, a confocal fluorescence microscope was used to observe whether or not the oil body carriers entered into the cells and the influence of the MOI value. The results show that the oil body carriers indeed entered into the cells, and the number of the oil body carriers entering into the cells also elevated with the increase of the MOI value. As shown in FIGS. 17 to 19 (in the figures, the binding percentage of cells and oil body carriers is defined as the number of cells binding to oil body carriers/10,000 cells×100), the above results conform to the analysis result from the flow cytometry.

### EXAMPLE 9

### Optimum Reaction Time of Oil Body Carriers and Tumor Cells

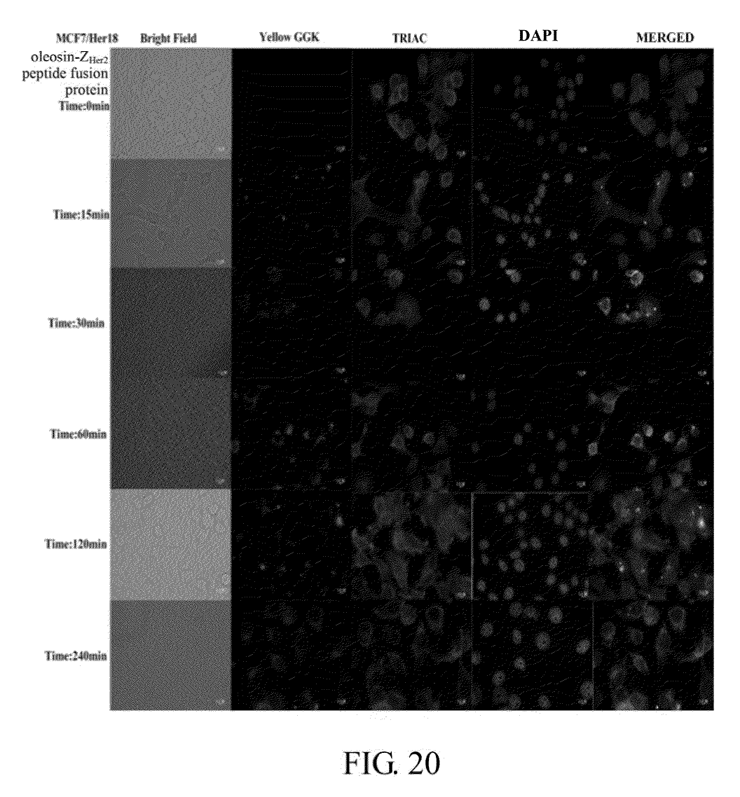

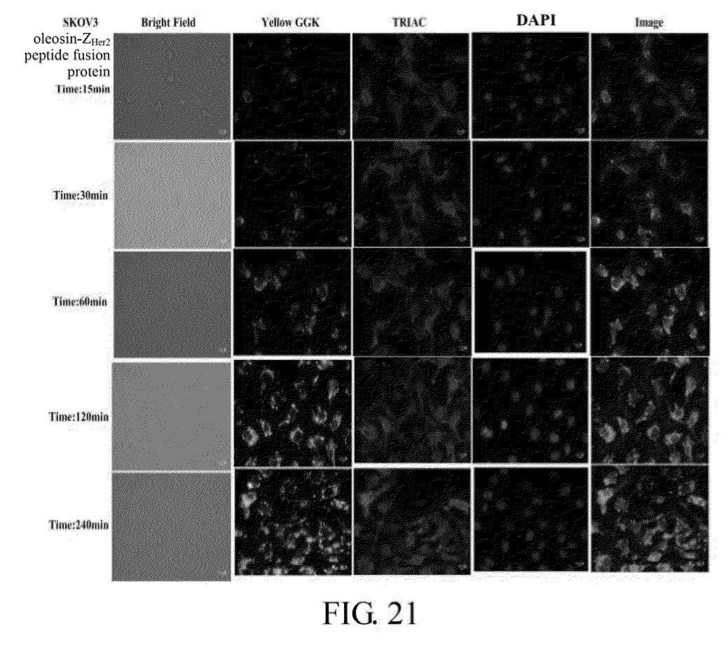

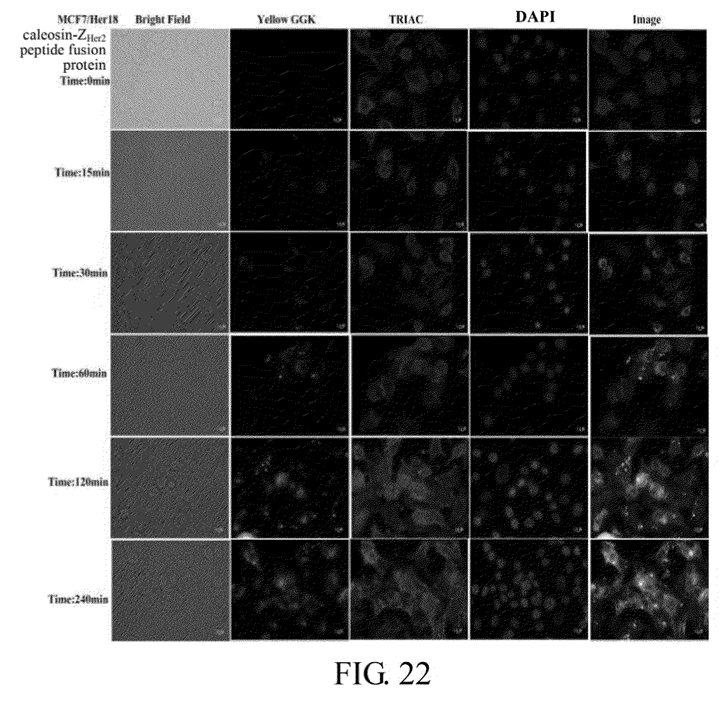

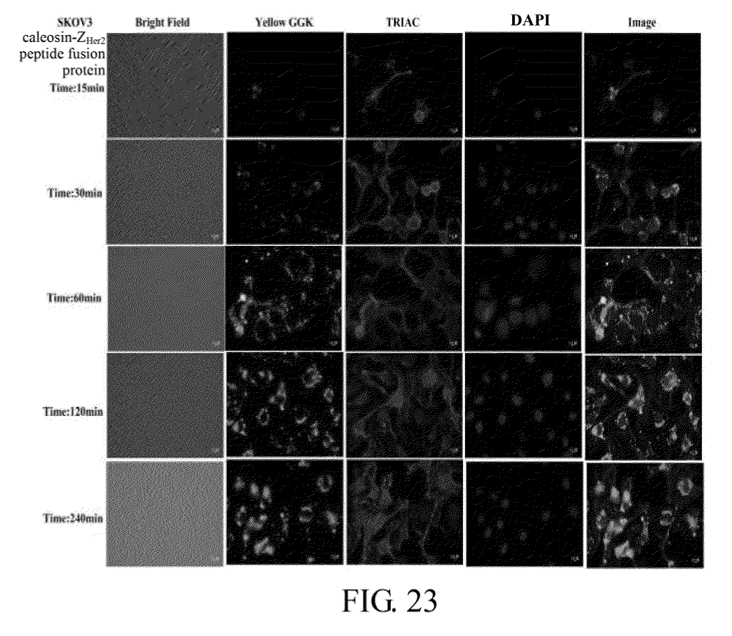

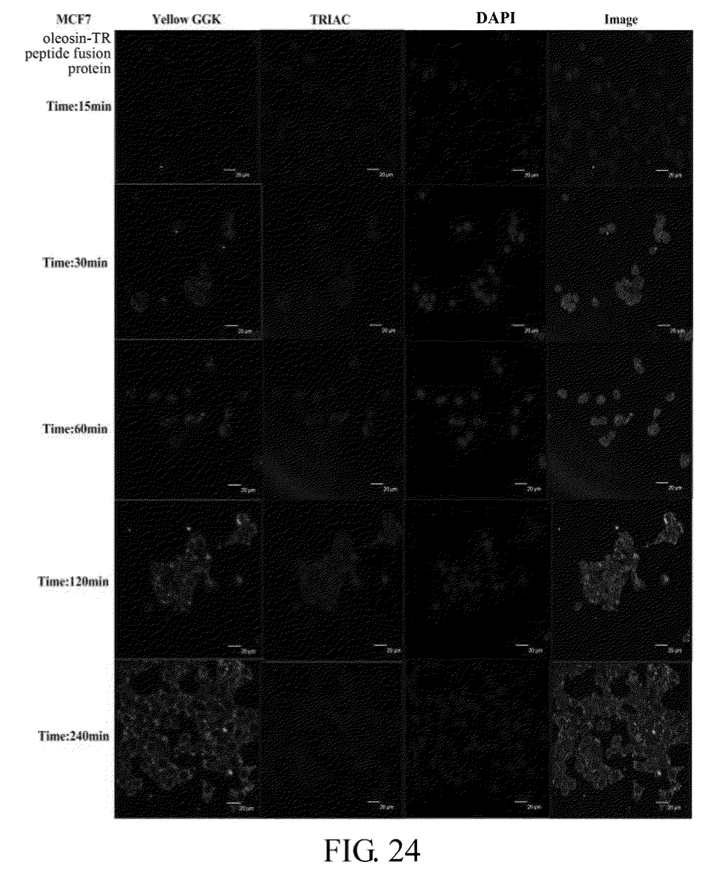

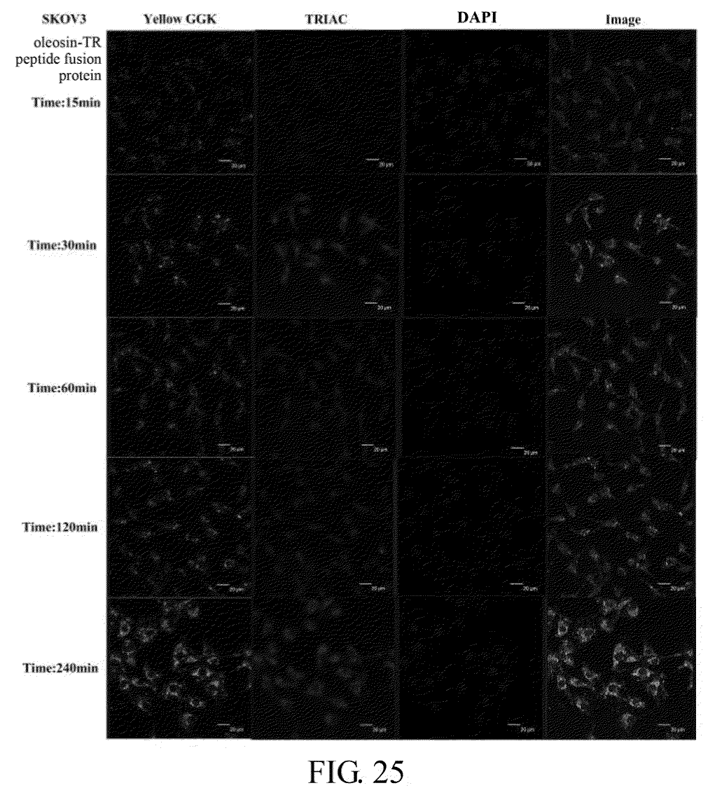

The optimum reaction time of the oil body carriers and tumor cells was determined by the same method as Example 8, and the MOI value was set at 200. The cell lines used were MCF7/Her18 cells and SKOV3 cells with overexpressing HER2/neu receptors, and MCF7 cells without overexpressing HER2/neu receptors, and the cells and the oil body carriers were reacted at different time points (0 to 240 minutes). The results are shown in FIGS. 20 to 25.

As shown in FIGS. 20 to 25, through observation under the fluorescence microscope, it was found that the oil body carriers comprising Z_{Her2} peptide specifically recognized the cells with overexpressing HER2/neu receptors, and the number of the oil body carriers entering into the cells elevated with the increase of the reaction time. The number reached its maximum when the reaction time was 2 hours.

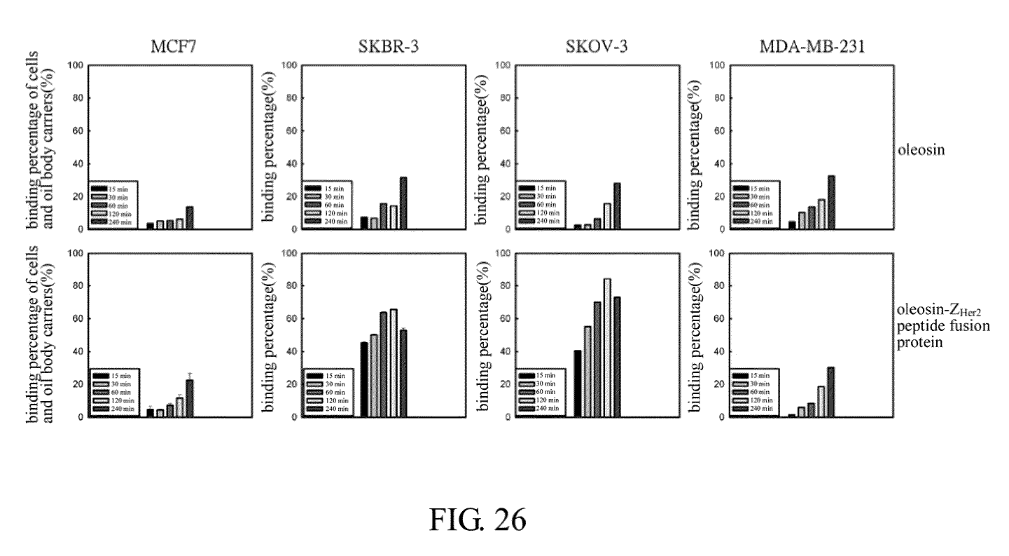

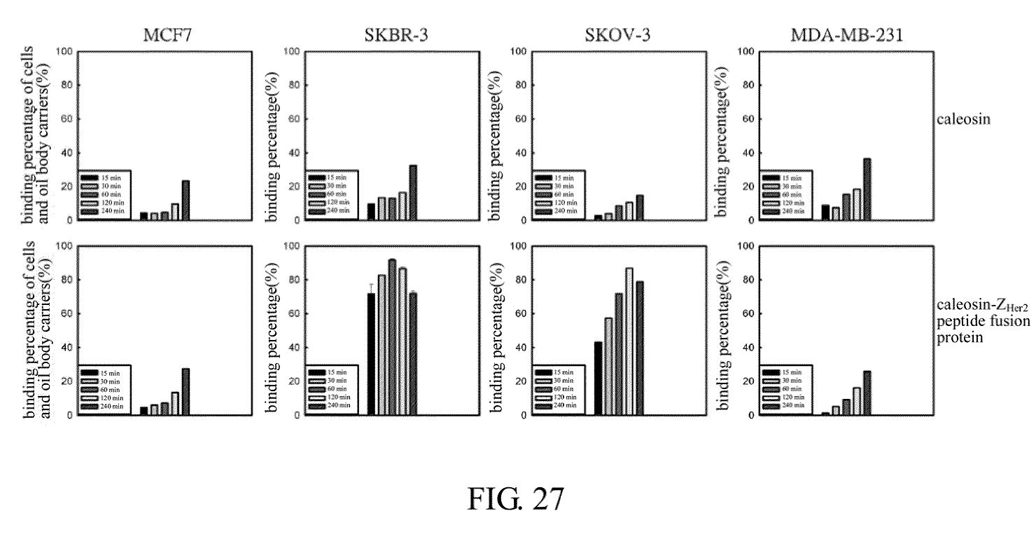

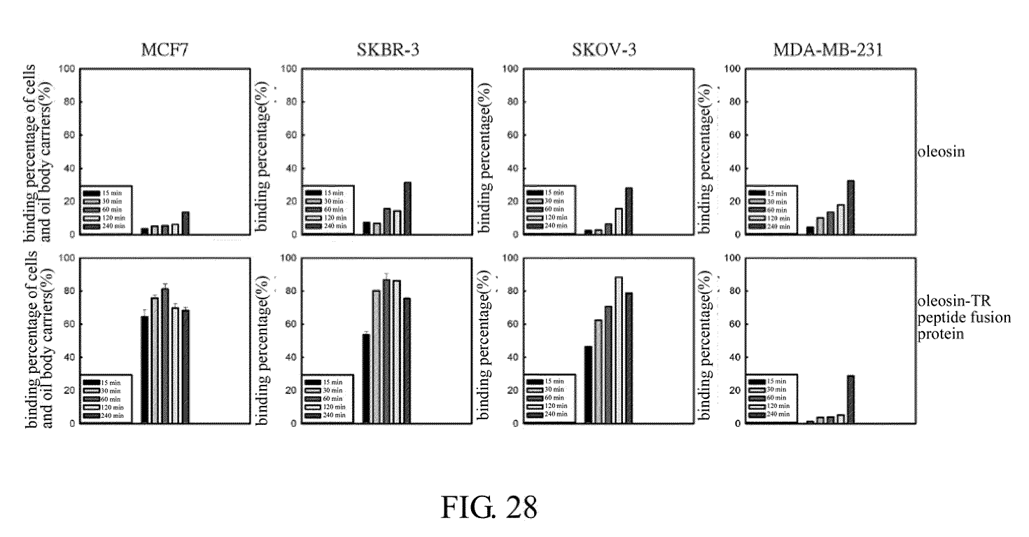

In addition, a confocal fluorescence microscope was used to observe whether or not the oil body carriers entered the cells and the influence of the reaction time. The results show that the number of the oil body carriers entering into the cells elevated with the increase of the reaction time, and the number reached its maximum when the reaction time was 2 hours. As shown in FIGS. 26 to 28 (in the figures, the binding percentage of cells and oil body carriers is defined as the number of cells binding to oil body carriers/10,000 cells×100), the above results conform the analysis result from the flow cytometry.

### EXAMPLE 10

### Target Therapy and Detection Assay-Cell Viability Test

First, according to the method in Example 1, oil body carriers covering (or comprising) anti-tumor drugs (lycopene or curcumin) with different concentrations (0 mM to 9 mM) were prepared as compositions for target therapy and/or detection.

Cells (MCF7 cells, MCF7/Her18 cells, SKOV3 cells, SKBR3 cells, or MDA-MV-231 cells) with a count of 5×10³ were inoculated in a 96-wells plate, and were incubated in a cell incubator (comprising 5 vol % carbon dioxide) at 37° C. for 24 hours. The next day, the above prepared compositions were added to the cells individually, and were reacted in an incubator for two hours. The medium was removed with a straw, and the cells were washed with PBS twice to remove unreacted oil body carriers. A fresh medium was then added to the cells, and after being incubated for 24, 48, 72, 96, and 120 hours, the cells were stained, and the number of live and dead cells was calculated under a microscope to observe if the composition inhibits cell growth, and to compare the biotoxicity of various compositions.

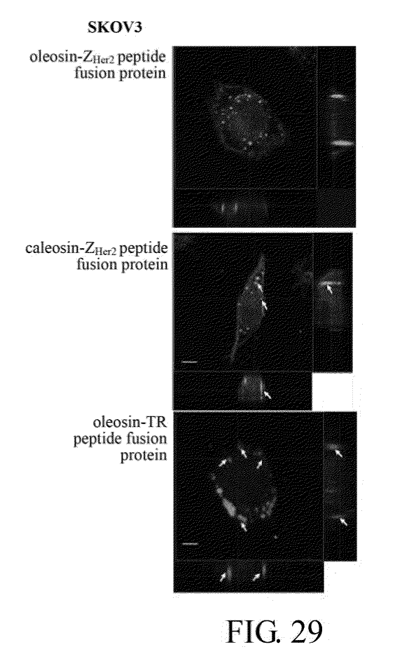

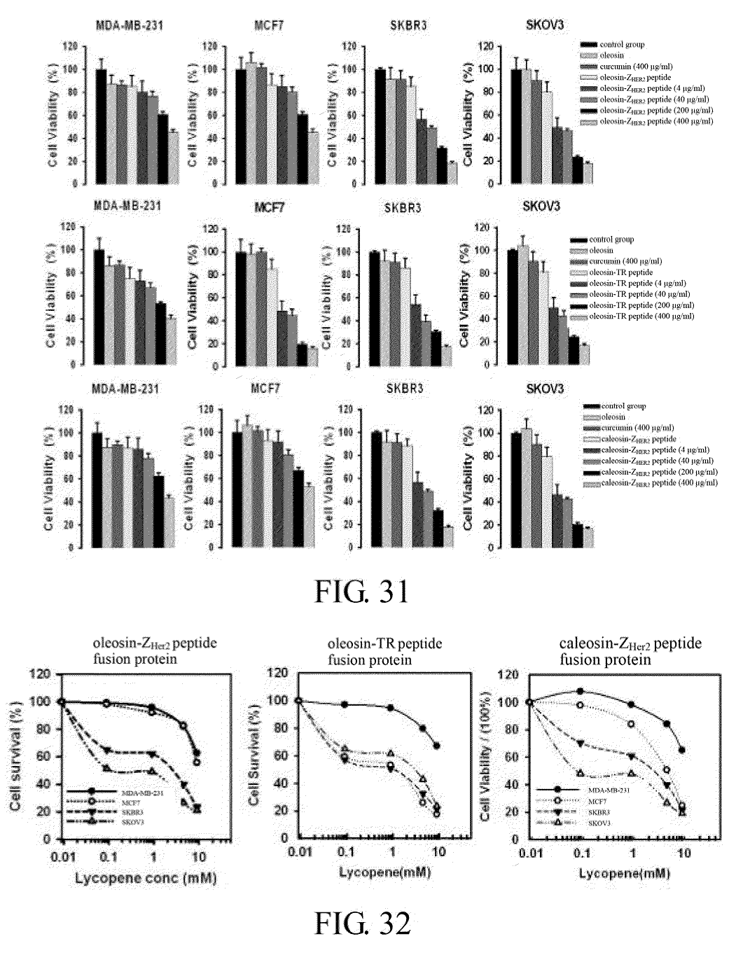

As shown in FIGS. 29 to 31, the oil body carriers of the present invention did not inhibit cell growth.

### EXAMPLE 11

### Target Therapy and Detection Assay-In Vitro Cell Test

Cells (MCF7 cells, MCF7/Her18 cells, SKOV3 cells, SKBR3 cells, or MDA-MV-231 cells) with a count of 5×10³ were inoculated in a 96-wells plate, and were incubated in a cell incubator (comprising 5 vol % carbon dioxide) at 37° C. for 24 hours. The next day, the compositions prepared in Example 10 were added to the cells individually, and were reacted in an incubator for two hours, and then the medium was removed with a straw, and the cells were washed with PBS twice to remove unreacted compositions. A fresh medium was then added to the cells, and after being incubated for 72 hours in the incubator, DMFM (comprising cell-counting kit-8 (CCK8), purchased from Dojindo Co.) was added to the cells and reacted for 2 hours. The absorbance at a wavelength of 450 nm was measured with an ELISA reader (purchased from DYNEX Technologies, Co.), and data was recorded. A standard curve was used to calculate the cell number.

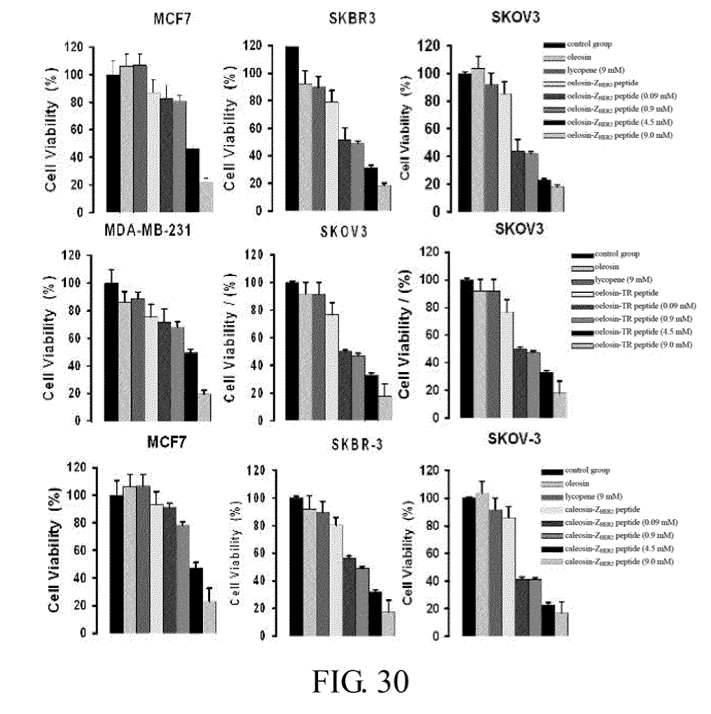

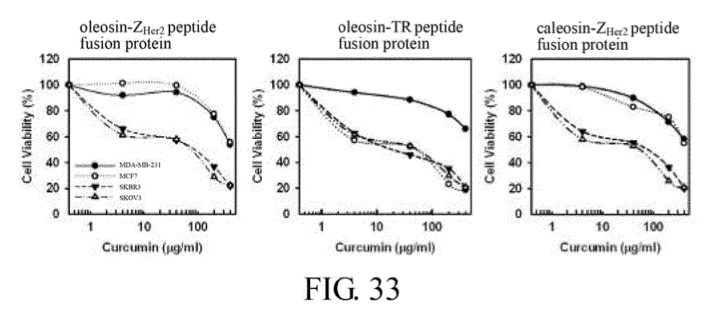

As shown in FIGS. 30 to 33, the compositions of the present invention efficiently inhibited tumor cell growth.

### EXAMPLE 12

### Target Therapy and Detection Assay-In Vivo Animal Test

Mice with breast cancer were used to conduct an animal test. Mice (BALB/c AnN.Cg-Foxn1nu/Cr1Nar1, purchased from National Laboratory Animal Center, Taiwan) were raised to an age of 3 to 8 weeks, and MDA-MB-231 or SKOV3 breast cancer cells were subcutaneously injected into the left dorsum of mice. The induction of breast cancer was conducted for about two weeks. After tumors generated by the breast cancer induction grew for four weeks, the size of the tumors was about 1,000 mm³, and the oil body carrier of the present invention (comprising 1.0 μg/ml-PBS oleosin-Z_{Her2} peptide, caleosin-Z_{Her2} peptide, or oleosin-TR peptide) was then injected into the mouse abdominal cavity. The mouse body was scanned at 1, 4, 8, and 24 hours with a 3D living body molecular image system (IVIS 200 System) to observe the blood circulation, breast cancer cell tracking, and the distribution in every organ of the oil body carrier in the mouse body.

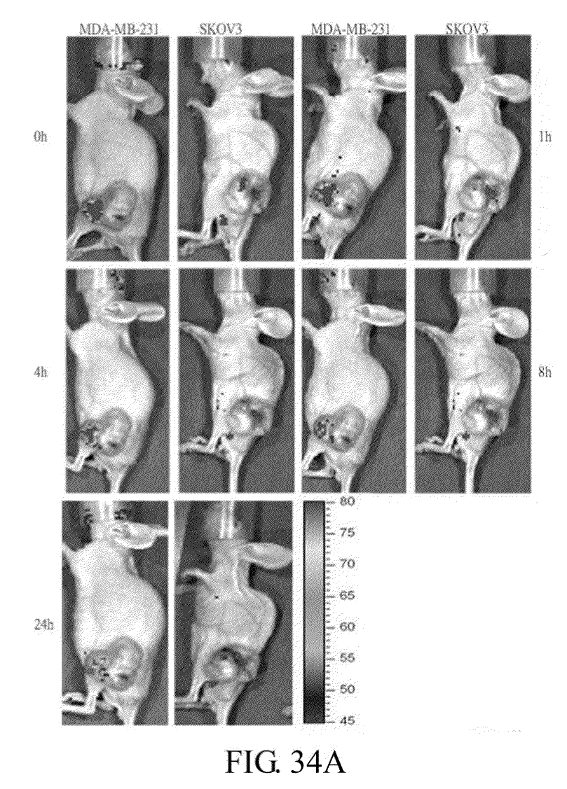

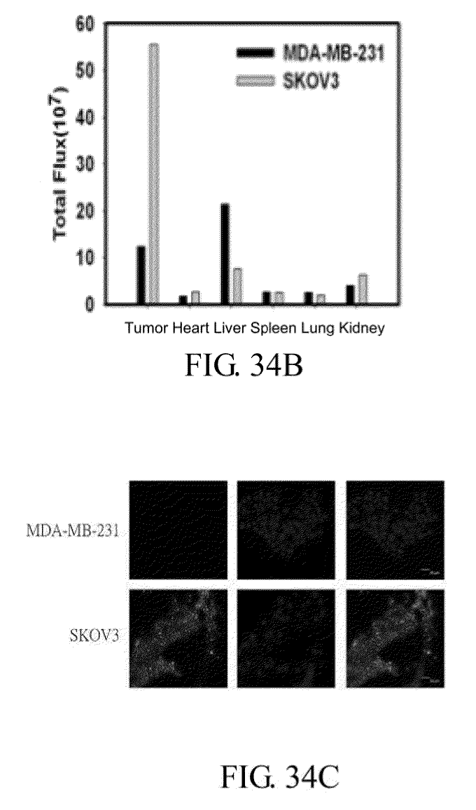

As shown in FIG. 34A, in the control group (the mice having MDA-MB-231 cells therein), the fluorescence emission in the mouse body disappeared gradually with the lapse of time; while in the experiment group (the mice having SKOV3 cells therein), clear fluorescence signals were still detected.

Then, the mice were anaesthetized with carbon dioxide and sacrificed by neck breaking. Tumors and organs were taken out for tissue slicing to observe the distribution of the oil body carriers. As shown in FIG. 34B, in the mouse body of the control group, the oil body carriers mostly accumulated in the liver (i.e., the organ where drug metabolism proceeds), indicating that the oil body carriers did not specifically enter into MAD-MB-231 tumor tissues; while in the mouse body of the experiment group, the oil body carriers stayed in the SKOV3 tumor tissues, indicating that the oil body carriers specifically tagged and entered into SKOV3 tumor tissues.

Furthermore, as shown in FIG. 34C, MAD-MB-231 tumor slices of the mice in the control group show that the distribution of the oil body carriers was unclear, while SKOV3 tumor slices of the mice in the experiment group show that the oil body carriers stayed in the tumor tissues.

### EXAMPLE 13

### Target Therapy and Detection Assay-Interaction Between Oil Body Carriers and Tumor Tissues

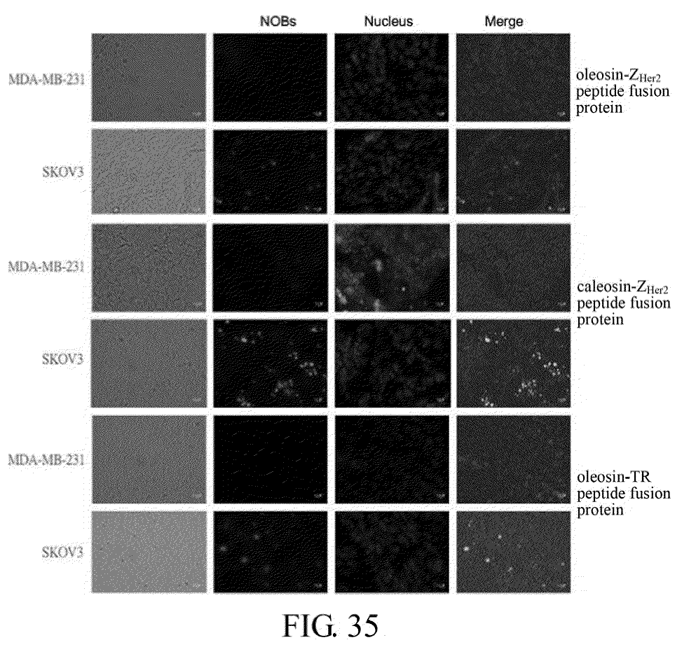

MAD-MB-231 and SKOV3 tumor tissues at the rear feet of the mice in Example 12 were taken out, and were embedded with OCT (a tissue freezing medium, purchased from LEICA Co.). The frozen section was then conducted with a freezing microtome (LEICA, CM3050S). A tissue slice was placed on a slide glass and washed with PBS for three times to remove OCT on the tissue, and tissue cells were fixed with a 2.5% formalin solution (comprising 0.5 g formalin powders, 2 ml 10X PBS, and 50 μl 5N sodium hydroxide) for 40 minutes. Then, the tissue slice was washed with PBS for three times to remove the remaining formalin solution thereon, and the oil body carrier (comprising 2.5 μg/ml-PBS oleosin-Z_{Her2} peptide) was added to react with the tissue cells for 120 minutes. After the reaction was completed, the tissue was washed with PBS three times to remove unreacted oil body carriers. The cell nuclei were then stained with DAPI in a proportion of 1:15,000 for five minutes. The tissue slice was washed with PBS three times to remove remaining DAPI, and the slice was sealed. The interaction between the oil body carriers and the tissue cells was observed with a fluorescence microscope. The results are shown in FIG. 35.

As can be seen in FIG. 35, in the MAD-MB-231 tumor slices of the control group, it was found that the oil body carriers did not tag or react with the tumor slices, while in the SKOV3 tumor slices of the experiment group, the oil body carrier comprising oleosin-Z_{Her2} peptide or caleosin-Z_{Her2} peptide fusion protein tagged the tumor tissue slices in which the cells had overexpressed HER2/neu receptors. The oil body carrier comprising oleosin-TR peptide fusion protein tagged the tumor tissue slices in which the cells had overexpressed α₅β₃ integrin receptors.

Based on the results in the above examples, it is shown that the average particle size of the oil body carrier of the present invention may successfully range from 10 to 2,000 nm. As described above, oil body carriers with such a particle size may achieve better drug delivery effect than conventional drug carriers with a larger particle size (e.g., liposomes, polymer particles, etc). In addition, the oil body carrier of the present invention has excellent stability in different lipids or at different pH values, and thus can be applied in target drug delivery and detection, or tagging of tumor cells, and has high utilization value in industries.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A fusion protein comprising:
a) an oil body protein, and
b) a peptide selected from a group consisting of a ligand peptide, an antibody peptide, a cell penetrating peptide, and combinations thereof.

2. The fusion protein as claimed in claim 1, wherein the oil body protein is from the seeds of a plant selected from a group consisting of sesame, olive, soybean, peanut, sunflower, mustard flower, flax, safflower, and combinations thereof.

3. The fusion protein as claimed in claim 1, wherein the oil body protein is an oleosin comprising an amino acid sequence of SEQ ID NO: 1 or a caleosin comprising an amino acid sequence of SEQ ID NO: 2.

4. The fusion protein as claimed in claim 3, wherein the oil body protein is from the seeds of sesame.

5. The fusion protein as claimed in claim 1, wherein the ligand peptide is a ligand peptide comprising an amino acid sequence of SEQ ID NO: 3 of a HER2/neu protein or a ligand peptide comprising an amino acid sequence of SEQ ID NO: 4 of an α₅β₃ integrin, and the cell penetrating peptide comprises an amino acid sequence of SEQ ID NO: 5.

6. A composition for target therapy and/or detection comprising:
a) an oil body carrier, comprising;
a1) a fusion protein, comprising an oil body protein and a peptide selected from a group consisting of a ligand peptide, an antibody peptide, a cell penetrating peptide, and combinations thereof, and
a2) a lipid, and
b) a medicament, a signal molecule, or a combination thereof,
wherein the weight/volume (μl/μl) ratio of the fusion protein and the lipid is at least about 1/25, and the average particle size of the oil body carrier is about 10 nm to about 2,000 nm.

7. The composition as claimed in claim 6, further comprising a buffer solution having a pH value of at least about pH 7.0.

8. The composition as claimed in claim 6, wherein the oil body protein is from the seeds of a plant selected from a group consisting of sesame, olive, soybean, peanut, sunflower, mustard flower, flax, safflower, and combinations thereof.

9. The composition as claimed in claim 6, wherein the oil body protein is an oleosin comprising an amino acid sequence of SEQ ID NO: 1 or a caleosin comprising an amino acid sequence of SEQ ID NO: 2.

10. The composition as claimed in claim 9, wherein the oil body protein is from the seeds of sesame.

11. The composition as claimed in claim 6, wherein the ligand peptide is a ligand peptide comprising an amino acid sequence of SEQ ID NO: 3 of a HER2/neu protein or a ligand peptide comprising an amino acid sequence of SEQ ID NO: 4 of an α₅β₃ integrin, and the cell penetrating peptide comprises an amino acid sequence of SEQ ID NO: 5.

12. The composition as claimed in claim 6, wherein the lipid is selected from a group consisting of triacylglycerol, olive oil, sesame oil, soybean oil, peanut oil, mineral oil, flax oil, safflower oil, and combinations thereof.

13. The composition as claimed in claim 12, wherein the lipid is sesame oil

14. The composition as claimed in claim 6, wherein the weight/volume ratio of the fusion protein and the lipid is about 1/1 to about 30/1.

15. The composition as claimed in claim 6, wherein the average particle size of the oil body carrier is about 10 nm to about 300 nm.

16. The composition as claimed in claim 7, wherein the buffer solution has a pH value of about 7.0 to about 9.0.

17. The composition as claimed in claim 6, wherein the medicament is selected from a group consisting of lycopene, curcumin, camptothecin, fat-soluble antibiotics, cucurbitacin, vinorelbine, and combinations thereof.

18. The composition as claimed in claim 6, wherein the signal molecule is selected from a group consisting of cadmium-cesium quantum dots, fluorescein isothiocyanate (FITC), Alizarine Yellow R (5-[(p-nitrophenyl)azo]salicylic acid sodium salt), Nile Red (9-diethylamino-5H-benzo[α]phenoxazine-5-one), and combinations thereof.

19. An oil body carrier comprising:
a) a fusion protein, comprising an oil body protein and a peptide selecting from a group consisting of a ligand peptide, an antibody peptide, a cell penetrating peptide, and combinations thereof, and
b) a lipid,
wherein the weight/volume (μg/μl) ratio of the fusion protein and the lipid is at least about 1/25, and the average particle size of the oil body carrier is about 10 nm to about 2,000 nm.

20. The oil body carrier as claimed in claim 19, further comprising a buffer solution having a pH value of at least about 7.0.
